(19) United States
(12) Patent Application Publication
(10) Pub. No.: US 2009/0068286 A1
(43) Pub. Date: Mar. 12, 2009

(54) METHOD OF TREATING CANCER BY ADMINISTRATION OF 5-SUBSTITUTED NUCLEOSIDES

(75) Inventors: Rudolf FAHRIG, Dresden (DE); Joerg-Christian Heinrich, Dresden (DE)

Correspondence Address: GREENBLUM & BERNSTEIN, P.L.C., 1950 ROLAND CLARKE PLACE, RESTON, VA, 20191, US

(73) Assignee: RESprotect, GmbH, Dresden (DE)

(21) Appl. No.: 11/853,540

(22) Filed: Sep. 11, 2007

(51) Int. Cl. A61K 31/70; A61K 33/24; A61P 35/00

(52) U.S. Cl. 424/649; 514/45

(57) ABSTRACT

The invention relates to methods of administration of at least one overexpression inhibitor of DNA repair genes and/or oncogenes (e.g., (E)-5-(2-bromovinyl)-2′-deoxyuridine (BVDU), or a prodrug, or salt thereof) to increase the cytotoxic effect of a cytostatic or cytotoxic chemotherapeutic agent during and/or after chemotherapy, e.g., in the treatment of cancer.

### FIELD OF THE INVENTION

The invention relates to the use of at least one overexpression inhibitor of DNA repair genes and/or oncogenes for producing a drug to increase the cytotoxic (e.g., apoptotic) effect of cytostatics after chemotherapy.

### BACKGROUND

Cancer diseases in humans are one of the most frequent causes of death and chemotherapy is the most frequent treatment method. The decrease in efficacy of chemotherapy over time is believed to be based on the occurrence of resistance.

Repeated treatment with cytostatic and/or cytotoxic drugs can lead to induction of chemoresistance and poor prognosis via a number of different mechanisms. Among these, such treatments can lead to overexpression in cancer cells of different survival pathways, of different DNA repair genes, of different oncogenes, and/or of uridine phosphorylase (UPase). Such treatments can also lead to overexpression of Stat3 and its target VEGF, which can lead to blockade of the initiation of anti-cancer immunity, enhancement of tumor cell proliferation, and/or prevention of apoptosis. Treatment with cytostatic and/or cytotoxic drugs can also lead to downregulation of DT-diaphorase and/or caspases. These factors, singly or in combination, can cause suppression of apoptosis and/or induction of chemoresistance. Suppression of apoptosis, in turn, can lead to genomic instability by recombination and other events and/or amplification of resistance genes like MDR and DHFR.

It is believed that these resistances have their root in the fact that cytostatic and/or cytotoxic drugs influence the expression of genes and have a genotoxic effect, i.e., induce mutations, gene amplifications and recombinations and hence destabilize the genome. In this way, chemotherapy induces or selects resistant cancer cells. Often oncogenes, such as e.g. Ras, Bc12, Bcr-abl, Myc, ErbB2 and others, are affected by such effects induced by cytostatics. Wrongly regulated expression of genes in conjunction with DNA repair and recombination also contributes to chemoresistance (e.g., p53 gene, BRCA1/2, UBE2N, APEX and Rad51). Furthermore enzymes which metabolize and bioactivate cytostatics (e.g. DHFR, DT-diaphorase (DT-D), or proteins which convey cytostatics (e.g. MDR1), which can lead to chemoresistance.

Most cytostatics eliminate tumor cells in that they induce apoptosis. Apoptosis is a form of programmed cell death which was described firstly in Kerr, J. F. et al., Br J Cancer, 26(4) (1972); 239-257 (incorporated by reference herein in its entirety). In contrast to necrosis, apoptosis is a physiological form of cell death. These two forms of cell death can be differentiated by means of differences between necrosis and apoptosis. Apoptosis has defined morphological and biochemical characteristics which occur successively as events of an ordered cascade. The continuous process can be divided into phases. Morphological characteristics of apoptosis are core chromatin condensation (karyopyknosis), shrinkage of cytoplasm, formation of apoptotic vesicles and finally apoptotic bodies. Tumor cells can prevent this by overactivation of survival mechanisms. Mechanisms of chemoresistance therefore also comprise anti-apoptotic acting genes, such as e.g. STAT3, the activated “signal transducer and activator of transcription 3” or JUN-D.

In 1995 effects of specific hormones and 5-substituted nucleosides which were hitherto unknown were discovered. These suppress the 2-amino-6-mercaptopurine (AMP)-induced SV40 amplification in cells of the Chinese hamster (Fahrig, R. et al., Mutat Res., 356 (2), 1996, 217-224) (incorporated by reference herein in its entirety) and triethylene melamine-induced recombination in yeasts (Fahrig, R., Mutat. Res., 372 (1), 1996, 133-139) (incorporated by reference herein in its entirety). In EP 0 806 956 B1 and U.S. Pat. No. 6,589,941 (incorporated by reference herein in their entireties), the treatment of leukemia cells of the mouse with 5-substituted nucleosides is described, the doxorubicin (adriamycin)-induced Mdr1 gene amplification and chemoresistance having been inhibited.

In the in vitro tests described above, 5-substituted nucleosides (i.e. base analogues) had always been applied together with one or more cytostatic or cytotoxic drugs.

U.S. Patent Publication 2006/0178338 (incorporated by reference herein in its entirety) discloses that 5-substituted nucleosides can increase cytostatic effects of chemotherapy even when administered after a cytotoxic cycle, that is, even when there is no cytotoxic remaining in a patient's system. The document provides in vitro experiments, as well as experiments in rats.

The prognosis of patients with certain cancers, including, without limitation, advanced pancreatic cancer, is very poor and effective treatments are lacking. In a pivotal study of gemcitabine (GEM), median survival was still less than 6 months (Glimelius B., et al., “Chemotherapy improves survival and quality of life in advanced pancreatic and biliary cancer.” Ann Oncol 1996; 7[6]:593-600) (incorporated by reference herein in its entirety). Co-treatment with gemcitabine and erlotinib, the second drug approved for pancreatic cancer, did not show a clinically significant effect. The effect of gemcitabine+erlotinib treatment vs. gemcitabine+placebo treatment showed increased overall survival (6.37 months vs. 5.9 months, p=0.025), improved one-year survival (24% vs. 17%) and improved time to progression (3.75 months vs. 3.55 months, p=0.003), while skin rash (6% vs. 1%) and diarrhea increased (6% vs. 2%).

There is a need for drugs and methods to prevent and/or delay the reduction in apoptotic effect caused by resistance formation in cancer treatment, including chemotherapy and/or cytotoxic therapy, and thereby to provide an effective and/or improved treatment method relative to the present state of the art.

There is a need for drugs and methods including the use of at least one overexpresssion inhibitor of DNA repair genes and/or oncongenes for producing a drug to increase the apoptotic effect of cytostatics after chemotherapy.

There is a need for drugs and methods to increase and/or improve one-year survival rates among cancer patients and/or to increase patient life span, including, without limitation, patients with pancreatic cancer.

### SUMMARY OF THE INVENTION

The present invention provides a method for treating a cancer treatable with gemcitabine comprising administering to a patient in need thereof a therapeutically effective amount of a cytotoxic composition comprising gemcitabine in a chemotherapy phase, and a therapeutically effective amount of a 5-substituted nucleoside, prodrug or salt thereof, or combination of two or more thereof, in at least one of the chemotherapy phase and a recovery phase. In a preferred embodiment, the cancer treatable with gemcitabine includes pancreatic cancer.

The present invention also provides a method for reducing chemoresistance comprising administering to a patient in need thereof a therapeutically effective amount of gemcitabine in a chemotherapy phase, and a therapeutically effective amount of a 5-substituted nucleoside, a prodrug or salt thereof, or combination of two or more thereof, in at least one of the chemotherapy phase or a recovery phase.

The present invention further provides a method for enhancing chemosensitivity comprising administering to a patient in need thereof a therapeutically effective amount of gemcitabine in a chemotherapy phase, and a therapeutically effective amount of a 5-substituted nucleoside, a prodrug or salt thereof, or combination of two or more thereof, in at least one of the chemotherapy phase or a recovery phase.

The present invention further provides a method for enhancing the cytotoxic effect of gemcitabine in treatment of pancreatic cancer, comprising administering a therapeutically effective amount of gemcitabine to a cancer patient in a chemotherapy phase, and a therapeutically effective amount of BVDU, or a prodrug, or salt thereof, in a recovery phase following the chemotherapy phase.

In preferred embodiments, methods of the present invention are used to treat patients with pancreatic cancer, and or to treat pancreatic cancer.

In a preferred embodiment, the BVDU, prodrug or salt thereof comprises the prodrug represented by the formula I:

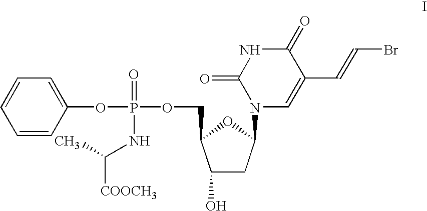

or a salt thereof.

In a preferred embodiment, the invention includes administering the 5-substituted nucleoside, prodrug or salt thereof, or combination of two or more thereof, during the chemotherapy phase. In a preferred embodiment, the chemotherapy phase is about 1 to 30 days, preferably about 1 to 5 days.

In a preferred embodiment, the invention includes administering the 5-substituted nucleoside, prodrug or salt thereof, or combination of two or more thereof, during the recovery phase. In a preferred embodiment, the recovery phase is about 1 to 10 days.

In a preferred embodiment, the method includes a rest phase during which no cytotoxic agent or 5-substituted nucleoside is administered. In a preferred embodiment, the rest phase is about 8 to 60 days.

In a preferred embodiment, the cytotoxic composition comprises a second cytotoxic agent other than gemcitabine. In a preferred embodiment, the second cytotoxic agent comprises a platinum compound, preferably cisplatin.

In a preferred embodiment, the 5-substituted nucleoside, prodrug, or salt thereof, or combination of two or more thereof, comprises BVDU, or a prodrug, or salt thereof.

In a preferred embodiment, the patient is administered the BVDU, prodrug, or salt thereof during the chemotherapy phase, and attains a BVDU blood level of about 0.02 to 50 μg/ml during the chemotherapy phase. Preferably, the patient attains a BVDU AUC of at least about 0.5 μg·hr/ml over a 24-hour period during the chemotherapy phase.

In a preferred embodiment the patient is administered the BVDU, prodrug, or salt thereof during the recovery phase, and attains a BVDU blood level of about 0.02 to 50 μg/ml during the recovery phase. Preferably, the patient attains a BVDU AUC of at least about 0.5 μg·hr/ml over a 24-hour period during the recovery phase.

The present invention provides drugs and methods that can increase, e.g., double, survival time. The invention provides drugs and methods for treatment of cancer, preferably pancreatic cancer, including the use of BVDU ((E)-5-(2-bromovinyl)-2′-deoxyuridine) with and/or after GEM, and more preferably in the use of BVDU with and/or after GEM+cisplatin (CIS).

### BRIEF DESCRIPTION OF THE DRAWINGS

The subject matter according to the invention is explained in more detail with reference to the following figures without limiting the subject matter to the mentioned embodiments.

FIG. 1 shows the effect of a cytostatic alone and in combination with BVDU on the number of AH13r cells.

FIG. 2 shows, in comparison to FIG. 1, the results with doxorubicin (DOX), mitoxantrone (MXA) and methotrexate (MTX).

FIG. 3 shows a Western Blot analysis for testing the “survival pathways” with doxorubicin (DOX).

FIG. 4 shows tests with mitomycin (MMC) corresponding to FIG. 3.

FIG. 5 shows the results of the measurement of DT-diaphorase (DT-D), doxorubicin (DOX) having been used alone or together with BVDU.

FIG. 6 shows tests with methotrexate (MTX) corresponding to FIG. 5.

FIG. 7 shows the course of disease followed by tumor marker CA19-9. Solid arrows indicate co-treatment BVDU+GEM+CIS; dashed arrows indicate second line therapy with different cytostatics alone.

FIG. 8 shows additional pilot study results. Median time to progression or median survival according to Box and Whisker and probability of time to progression or of survival according to Kaplan-Meier. Upper line (left graph) and bar “a” (right graph) represent BVDU co-treatment group; lower line (left graph) and bar “b” (right graph) represent chemotherapy alone group; the x's represent one patient without progression and three living patients, respectively.

FIG. 9 shows median survival according to Box and Whisker and probability of time to progression or of survival according to Kaplan-Meier. “X's” represent persons without progression or living patients.

FIG. 10 shows pharmacokinetic results. GEM: The maximum concentrations were measured immediately after completion of the 30 min infusion. BVDU is given half an hour before GEM and the second time 4 hours later. GEM disappeared within this time. Therefore, an influence on GEM concentration by BVDU could only be exerted by the first intake of tablets. This leads to the result that only two different doses of BVDU (125 and 250 mg) can be compared. BVDU: All measurement is performed within 9 hours. This means that only the first three intakes of tablets were relevant. In dose groups four and five the intake of tablets was identical.

FIGS. 11a and b illustrate examples of chemotherapy, recovery and treatment phases in pancreas cancer patients.

FIG. 12 shows effect of BVDU alone on tumor size in rats.

FIGS. 13a-d illustrates, without limitation, various treatment schedules.

FIG. 14 illustrates effect of BVDU and cisplatin, with no BVDU in the recovery phase, on tumor size in rats.

FIG. 15 shows effects of BVDU and doxorubicin on tumor size in rats.

FIG. 16 shows effects of BVDU and cisplatin on tumor size in rats.

FIG. 17 shows effects of BVDU and cyclophosphamide on tumor size in rats.

### DETAILED DESCRIPTION OF THE INVENTION

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is “from about 1 to 50” (or equivalently, “from about 1 to about 50”), it is deemed to include, for example, 1, 7, 34, 46.1, 23.7, or any other value or range within the range.

Similarly, when a parameter, variable, or other quantity, is described with a set of upper values, and a set of lower values, then this is to be understood as an express disclosure of all ranges formed from each pair of upper and lower values.

5-substituted nucleosides useful in the present invention include any 5-substituted nucleoside that shows an anti-cancer effect when administered with and/or after a cytotoxic treatment cycle. Preferred 5-substituted nucleosides include 5-substituted uridines and derivatives thereof, preferably 5-substituted 2′-deoxyuridines, more preferably BVDU (also known as RP101). Included within this description are protected forms, prodrugs and salts thereof, as well as combinations of one or more thereof. The 5-substituted nucleosides of the present invention are generally referred to as “overexpression inhibitors.”

As the term is used herein, “prodrug” refers to any compound that is metabolized, directly or indirectly, into a 5-substituted nucleoside that shows an anti-cancer effect when administered with and/or after a cytotoxic treatment cycle. Any prodrug can be used within the present invention. An example of a prodrug of BVDU according to the invention is represented in the general formula I:

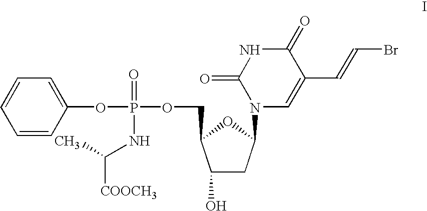

As is understood in the art, and as used herein, the terms “protected form” and “prodrug” are interchangeable, the former being predominantly used by chemists, and the latter being predominantly used by pharmacists. Without altering the scope in any regard, the present disclosure will generally employ the term “prodrug” to cover both usages.

When a salt form is used, any pharmaceutically acceptable salt may be used. Such salts include, but are not limited to acetate, adipate, alginate, arginate, aspartate, benzoate, benzenesulfonate (besylate), bisulfate, bisulfite, bromide, butyrate, citrate, camphorate, camphorsulfonate, caprylate, chloride, chlorobenzoate, citrate, cyclopentanepropionate, digluconate, dihydrogenphosphate, dinitrobenzoate, dodecysulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate (mesylate), methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, phthalate, picrate, pivalate, propionate, salicylate, sodium phosphate, stearate, succinate, sulfate, sulfosalicylate, tartrate, thiocyanate, thiomalate, tosylate, and undecanoate, as well as mixtures and combinations of two or more thereof.

The present invention includes use and/or administration of a therapeutically effective amount of a 5-substituted nucleoside, prodrug or salt thereof, or combination of two or more thereof. It should be understood that when a combination of two or more is used, that it is the combination that is therapeutically effective, and not necessarily the amount of any particular component of the combination. In a combination, particular components may be present in amounts that would be sub-therapeutic if used or administered in isolation.

It is surprisingly observed that effectiveness of BVDU appears to be independent of the cytostatic drug used or the type of tumor. The present invention can be used with any cancer treatment regime, including radiation therapy and chemotherapy, preferably chemotherapy. As used herein, the terms “cytostatic” and “cytotoxic” will be used interchangeably, and both refer to the active ingredient used in a chemotherapy regime, which active agent may be a single chemical entity, or a combination of two or more entities.

Cytostatic agents that can be used according to the present invention include agents used for treatment of any type of cancer, including solid and non-solid tumors. These include, without limitation, sarcomas, leukemias, adenocarcinomas, and cancers of the pancreas, lung (including small cell and non-small cell lung cancer), colon, breast, prostate, cervix, liver, brain, bone, ovary, esophagus, stomach and skin. These include agents used against primary tumors, as well as agents used against non-primary cancers, e.g., metastasized tumors, and further include agents characterized as first-line agents, as well as second-line or other agents. The terms, “cytostatic agents”, “cytostatics” and “cytotoxic agent” are used interchangeably herein to refer to agents used for treatment of cancer of any type, including solid and non-solid tumors. “Cytostatic agents”, “cytostatics” and “cytotoxic agent” are also referred to herein as “anti-cancer agents”.

As used herein, the term “effective” means that the cytostatic agent in question is effective, in a statistical sense, at treating the type of cancer. Effectiveness can be assessed by any measure useful for determining effectiveness. Such measures can include, without limitation, half-year or one-year survival rate, median survival, overall survival and/or time to progression (TTP). The term “effective” does not, however, mean that the particular agent was, is, or will be, effective in a particular individual. As used herein, the term “TTP” refers to an interval of time from when a disease is diagnosed (or treated) until the disease worsens, e.g., progresses to the next stage. The term “TTP” can also refer to the length of time a disease spends in a particular stage of progression.

Cytostatic agents that can be used according to the present invention include agents that work through any mechanism of action. Cytostatic agents used according to the present invention include agents that target cells undergoing DNA synthesis, agents that block progression through the G1/S-phase boundary, DNA damaging or intercalating agents, spindle poisons and combinations of two or more thereof.

It is believed that any class of cytostatic agent can be used according to the present invention. Some classes of cytostatic agents that can be used include, without limitation, alkylating agents, anti-metabolites, antibiotics, platinum agents, and combinations of two or more thereof. Some of these include, without limitation, gemcitabine, platinum-based drugs (e.g., cisplatin), etoposide, doxorubicin, vinorelbin, irinotecan, cyclophosphamide, epirubicin, paclitaxel, docetaxel, vincristine, mitoxantrone, mitomycin C, erlotinib, mitoxantrone and methotrexate, and combinations of two or more thereof.

In a preferred embodiment, the present invention can be used to treat cancers that are treatable with gemcitabine. By this is meant that gemcitabine, alone or in combination with one or more other anti-cancer agents, can be used to treat the cancer. The term is used in a statistical sense, and does not imply that a given individual will be successfully treated. Such cancers include, without limitation, pancreatic cancer, prostate cancer, small cell and non-small cell lung cancer, breast cancer, and ovarian cancer, preferably pancreatic cancer.

Cytostatics can be used singly or in combination, i.e., a cytostatic composition according to the present invention can include one or more cytostatic agents. Moreover, a cytostatic composition need not be a single composition, but can include two or more compositions, which can be administered via the same or different route of administration (e.g., IV and/or oral tablet). For example, gemcitabine and cisplatin may be administered to a patient via two or more IV containers, and via two or more injection sites. In this example, gemcitabine and cisplatin would be considered, together, to be a cytotoxic composition.

In general, chemotherapy cycles according to the present invention can be organized in three phases, which will be referred to herein as a chemotherapy phase, a recovery phase, and a rest phase. The only phase in which a cytotoxic agent is administered is the chemotherapy stage, during which a 5-substituted nucleoside may optionally be administered, without limitation, concurrently with, or prior to, administration of the cytotoxic agent. The recovery phase begins after the chemotherapy phase, preferably immediately after. During the recovery phase, no cytotoxic agent is administered, but the recovery phase includes administration of at least one 5-substituted nucleoside, preferably BVDU (or a prodrug, or salt thereof). During the rest phase, no cytotoxic or 5-substituted nucleoside is administered. While the chemotherapy and recovery phases are described above by administration (or lack of administration) of certain agents, it should be understood that the phases also include periods of time after administration, during which the administered active is still present in therapeutic levels in the patient's system, e.g., in a patient's blood. During the rest phase, typically no cytotoxic or 5-substituted nucleoside is present at therapeutic levels in a patient's system, e.g., in a patient's blood.

The lengths and patterns of the three phases will depend on a number of factors, such as the cytotoxic agent(s) used, the type and stage of the cancer being treated, and the condition of the patient being treated. The treatment cycles are generally the same in all three clinical study phases, though they may be different. A patient who lives long enough can be treated with several treatment cycles (e.g., 2, 3, 4, 5, 6, 7, 8, or more than 8) of the cytotoxic drugs.

Any length chemotherapy phase, recovery phase, and/or rest phase may be used in the present invention. A chemotherapy phase, e.g., days in which a chemotherapy agent is administered, will generally be, without limitation, at least about 1, 2, 3, 4, or 5 days long, and generally less than, or about 14, 10, or 7 days long. A recovery phase will generally be, without limitation, at least about 2, 3, 4, 5 or 7 days long, and generally less than about one month (e.g., 30 or 31 days), 4, 3, 2 weeks, 10 or 7 days long. A rest phase will generally be, without limitation, at least about 1 week, 10 days, 2, 3, 4 or 8 weeks long, 1, 2, 3 months long, along with ranges formed from combinations of these times.

A “treatment phase” can be considered as the combination of chemotherapy phase and recovery phase. In general, without limitation, a treatment phase can be about, or longer than about, 2, 3, or 4-8 treatments within 18, 19 or 30 days. For example, two treatments in 19 days (2×1 treatments within 19 days), three treatments in 18 days (3×1 treatment within 18 days), 4 to 8 treatments within 30 days, or 1 to 30 treatments within 30 days. The first two of these treatment phases are illustrated in FIG. 11. An entire chemotherapy cycle, including all chemotherapy, recovery, and rest phases, will generally be about or less than about 8, 18, 30, 40, 60 or up to 700 days. Without limitation, ranges include, e.g., about 8-700 days.

In general, a recovery phase can be about, or longer than about two recovery phases of four days each over a 19 day period (2×4 days within 19 days) and afterwards a rest phase of at least 10 days before the next cycle starts; three recovery phases of three days each over an 18-day period (3×3 days within 18 days) and afterwards a rest phase of at least 10 days before the next cycle starts; 3 to 10 days within 18 days and afterwards a rest phase of up to 60 days before the next cycle starts, or 10 to 60 days recovery phase. A recovery phase will generally be about or less than 1, 2, 3, 4, 5, 10, 20, 30 or up to 60 days. Thus, ranges for recovery phases include, e.g., about 5-20 days, or 1-60 days.

The 5-substituted nucleoside may be present at or above any amount in the blood that is effective. Amounts should not be so high as to induce unacceptable side effects, although drugs might be used to ameliorate side effects. Levels of 5-substituted nucleoside need not be the same in the recovery phase as in the chemotherapy phase. Indeed, because of the absence of chemotherapy agents (which can induce strong side effects) in the recovery phase, it might be possible to employ higher levels of 5-substituted nucleosides during the recovery phase than in the chemotherapy phase. Higher levels of 5-substituted nucleosides during the chemotherapy phase than in the recovery phase are also contemplated in this invention.

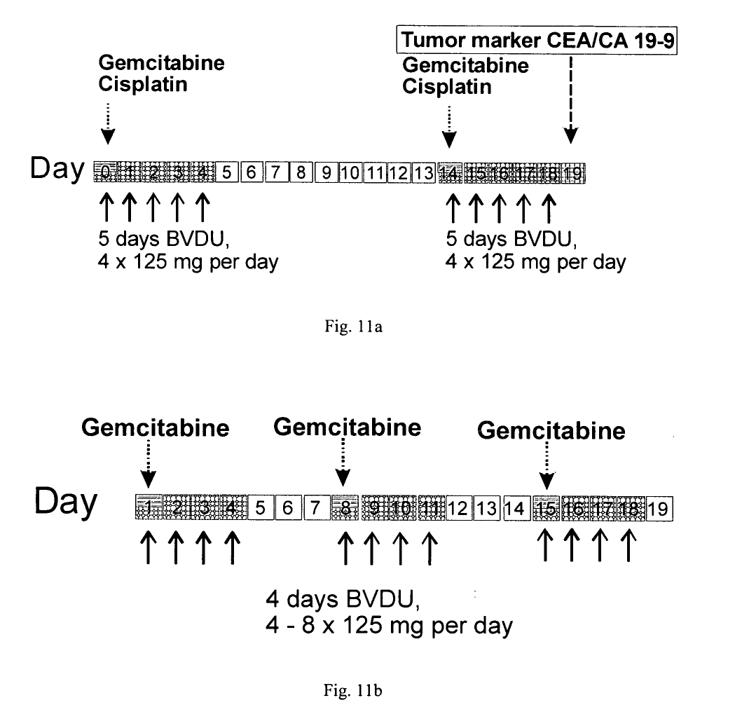

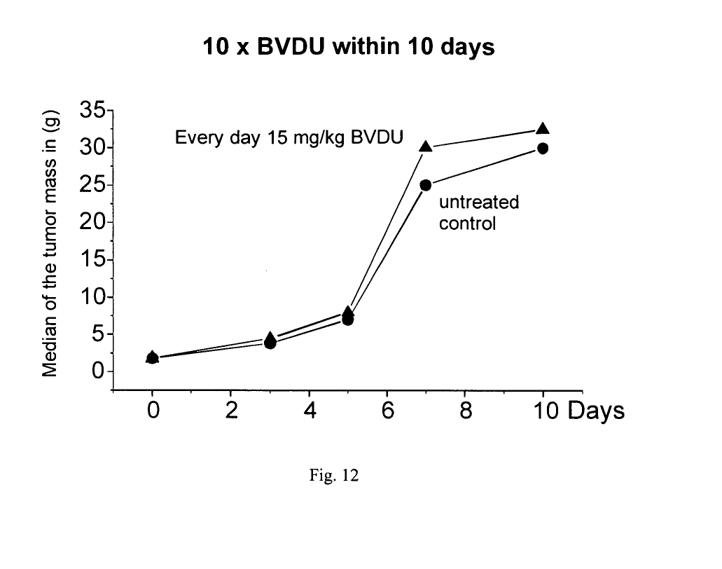

When BVDU is used (or a prodrug, or salt thereof), BVDU may be present in at least a therapeutically-effective amount, preferably a blood concentration of BVDU of at least about 0.02 μg/ml, more preferably at least about 0.05 μg/ml, more preferably at least about 0.5 μg/ml. It is expected that blood levels may be kept below about 50 μg/ml, or below about 20, or about 10, or about 5 μg/ml. Ranges may be defined (here and throughout this document) by any combination of upper and lower bounds, e.g., about 0.05 to 20 μg/ml. FIG. 12 illustrates some BVDU blood levels obtained from patients in clinical trials. The data on which FIG. 12 is based are presented in Tables 1 and 2, below. Table 1 shows day-1 blood sampling and dosing schedules for BVDU and gemcitabine. Table 2 shows plasma concentrations of BVDU (ng/mL) obtained after oral administration of BVDU (day-1 values).

TABLE 1

| Time | Drug | Gemcitabine | BVDU |
|---|---|---|---|
| (hr) | Administered | Sample | Sample |
| Pre-dose | — | X | X |
| 0 | BVDU | — | — |
| 0.5 | — | — | X |
| 1.0 | Start of Gemzar | X | X |
| | infusion | | |
| 1.5 | End of Gemzar | X | — |
| | infusion | | |
| 2.0 | — | X | X |
| 3.0 | — | X | X |
| 4.0 | BVDU | — | X |
| 5.0 | — | X | X |
| 8.0 | BVDU | — | — |
| 9.0 | — | X | X |
| 12.0 | BVDU | — | — |

TABLE 2

| BVDU | | | | | | | | | | |
|---|---|---|---|---|---|---|---|---|---|---|
| Dose | | Time (hr) | | | | | | | | |
| Level (mg) | Patient | 0.0 | 0.5 | 1.0 | 1.5 | 2.0 | 3.0 | 4.0 | 5.0 | 9.0 |
| 6000 | 101 | 0 | 320 | 339 | 226 | 207 | 133 | 126 | 280 | 298 |
| | 102 | 0 | — | — | — | — | — | — | 73 | 138 |
| | 103 | 0 | — | 109 | 411 | 366 | 277 | 122 | 458 | 508 |
| | 104 | 0 | 106 | 82 | 114 | 120 | 116 | 90 | 388 | 620 |
| | N | 4 | 2 | 3 | 3 | 3 | 3 | 3 | 4 | 4 |
| | Mean | 0 | 213 | 177 | 250 | 231 | 175 | 113 | 300 | 391 |
| | SD | 0 | 151 | 141 | 150 | 125 | 88 | 20 | 168 | 215 |
| | Min | 0 | 106 | 82 | 114 | 120 | 116 | 90 | 73 | 138 |
| | Median | 0 | 213 | 109 | 226 | 207 | 133 | 122 | 334 | 403 |
| | Max | 0 | 320 | 339 | 411 | 366 | 277 | 126 | 458 | 620 |
| | CV % | — | 71.0 | 79.9 | 59.9 | 54.0 | 50.4 | 17.5 | 56.0 | 55.0 |
| 7500 | 105 | 0 | 1126 | 1182 | 728 | 792 | 444 | 242 | 280 | 1986 |
| | 106 | 0 | 132 | 224 | 196 | 227 | 355 | 418 | 776 | 360 |
| | 107 | 0 | 1806 | 464 | 358 | 261 | 207 | 178 | 814 | 1086 |
| | 108 | 0 | 380 | 484 | 848 | 586 | 198 | 112 | 188 | 428 |
| | 201 | 0 | 4574 | 2008 | 644 | 403 | 304 | 264 | 1146 | 898 |
| | N | 5 | 5 | 5 | 5 | 5 | 5 | 5 | 5 | 5 |
| | Mean | 0 | 1604 | 872 | 555 | 454 | 302 | 243 | 641 | 952 |
| | SD | 0 | 1786 | 729 | 270 | 236 | 103 | 115 | 400 | 655 |
| | Min | 0 | 132 | 224 | 196 | 227 | 198 | 112 | 188 | 360 |
| | Median | 0 | 1126 | 484 | 644 | 403 | 304 | 242 | 776 | 898 |
| | Max | 0 | 4574 | 2008 | 848 | 792 | 444 | 418 | 1146 | 1986 |
| | CV % | — | 111.4 | 83.5 | 48.6 | 52.0 | 34.3 | 47.2 | 62.4 | 68.8 |
| 9000 | 109 | 0 | 440 | 248 | 62 | 610 | 693 | 726 | 794 | 348 |
| | 110 | 0 | 234 | 93 | 160 | 366 | 282 | 455 | 484 | 1636 |
| | 111 | 0 | 1518 | 1314 | 900 | 799 | 577 | 300 | 2690 | 2420 |
| | 202 | 0 | 1574 | 1480 | 1666 | 1029 | 304 | 198 | 1246 | 1630 |
| | N | 4 | 4 | 4 | 4 | 4 | 4 | 4 | 4 | 4 |
| | Mean | 0 | 942 | 784 | 697 | 701 | 464 | 420 | 1304 | 1509 |
| | SD | 0 | 703 | 714 | 746 | 281 | 203 | 230 | 976 | 858 |
| | Min | 0 | 234 | 93 | 62 | 366 | 282 | 198 | 484 | 348 |
| | Median | 0 | 979 | 781 | 530 | 705 | 441 | 378 | 1020 | 1633 |
| | Max | 0 | 1574 | 1480 | 1666 | 1029 | 693 | 726 | 2690 | 2420 |
| | CV % | — | 74.7 | 91.1 | 107.1 | 40.2 | 43.8 | 54.8 | 74.9 | 56.9 |
| 10500 | 112 | — | 188 | 420 | 506 | 1136 | 308 | 130 | 410 | 1150 |
| | 203 | 0 | 2202 | 1648 | 868 | 669 | 228 | 88 | 202 | 1084 |
| | 204 | 0 | 290 | 1012 | 138 | 122 | — | — | 240 | 1386 |
| | 301 | 0 | 1722 | 1058 | 1100 | 1067 | 249 | 294 | 448 | 1934 |
| | 302 | 0 | 1154 | 916 | 566 | 490 | 1026 | 310 | 702 | 4788 |
| | N | 4 | 5 | 5 | 5 | 5 | 4 | 4 | 5 | 5 |
| | Mean | 0 | 1111 | 1011 | 636 | 697 | 453 | 206 | 400 | 2068 |
| | SD | 0 | 879 | 438 | 367 | 419 | 384 | 113 | 199 | 1557 |
| | Min | 0 | 188 | 420 | 138 | 122 | 228 | 88 | 202 | 1084 |
| | Median | 0 | 1154 | 1012 | 566 | 669 | 279 | 212 | 410 | 1386 |
| | Max | 0 | 2202 | 1648 | 1100 | 1136 | 1026 | 310 | 702 | 4788 |
| | CV % | — | 79.1 | 43.3 | 57.7 | 60.2 | 84.7 | 55.0 | 49.7 | 75.3 |
| 12000 | 205 | 0 | 3334 | 1826 | 844 | 561 | 407 | 440 | 1074 | 1778 |
| | 206 | 0 | 672 | 1018 | 1524 | 1788 | 1540 | 796 | 4958 | 4038 |
| | 303 | 0 | 452 | 647 | 792 | 362 | 268 | 392 | 200 | 1220 |
| | N | 3 | 3 | 3 | 3 | 3 | 3 | 3 | 3 | 3 |
| | Mean | 0 | 1486 | 1164 | 1053 | 904 | 738 | 543 | 2077 | 2345 |
| | SD | 0 | 1604 | 603 | 408 | 772 | 698 | 221 | 2533 | 1492 |
| | Min | 0 | 452 | 647 | 792 | 362 | 268 | 392 | 200 | 1220 |
| | Median | 0 | 672 | 1018 | 844 | 561 | 407 | 440 | 1074 | 1778 |
| | Max | 0 | 3334 | 1826 | 1524 | 1788 | 1540 | 796 | 4958 | 4038 |
| | CV % | — | 108.0 | 51.8 | 38.8 | 85.5 | 94.5 | 40.7 | 121.9 | 63.6 |

When a combination of two or more 5-substituted nucleosides, or prodrugs or salts thereof, is used or administered, the combination may be administered in any way, including, without limitation, orally (including, without limitation, via tablet, capsule, or orally disintegrating form), parenterally (including, without limitation, intravenously, via bolus injection or slow infusion), or by other means (including, without limitation, transdermally). The components of a combination may be administered in the same dosage form (e.g., all components present in a single tablet), in different dosage forms (e.g., different components among two or more tablets), or by different route of administration (e.g., different components administered parenterally and orally), and they may be administered simultaneously, or sequentially. Administration is preferably oral. Dosage forms are preferably oral dosage forms, more preferably tablets. Dosage forms for combinations preferably include all 5-substituted nucleosides, prodrugs and/or salts thereof, in a single dosage form, preferably an oral dosage form, preferably a capsule or tablet.

BVDU, or prodrug or salt thereof, may be administered in any way, and by any route of administration, via any dosage form, that results in obtaining a therapeutically effective amount of BVDU in a patient. BVDU, or prodrug or salt thereof, may be administered orally (including, without limitation, via tablet, capsule, or orally disintegrating form), parenterally (including, without limitation, intravenously, via bolus injection or slow infusion), or by other means (including, without limitation, transdermally). Combinations of two or more dosage forms and/or routes of administration are also included in the present invention. Administration is preferably oral. Dosage forms are preferably oral dosage forms, more preferably tablets.

Any amount of BVDU that results in effective blood levels can be used in accordance with this invention, and may depend on the size and condition of the patient. BVDU is preferably administered in amounts greater than, or about, 100, 250 or 500 mg daily. BVDU is preferably administered in amounts less than, or about 3000, 2000, or 1500 mg daily. Some representative non-limiting levels of BVDU administered per day include amounts of, or about, e.g., 500, 625, 750, 875, 1000 or 1500 mg daily.

In order to maintain effective blood levels, the 5-substituted nucleoside, e.g., BVDU, is preferably administered over 3, 4 or 5, preferably 4, divided doses daily. Representative doses of BVDU include 500 mg daily, which can be administered as, e.g., 4 divided doses of 125 mg each (4×125 mg); 625 mg, which can be administered as, e.g., 1×250 mg+3×125 mg daily; 750 mg, which can be administered as, e.g., 2×250 mg+2×125 mg daily; 875 mg, which can be administered as, e.g., 3×250 mg+1×125 mg daily; or 1000 mg, which can be administered as, e.g., 4×250 mg daily. The dosage can also be increased over a chemotherapy and/or recovery phase, or can be increased from one recovery phase to another. Thus, for example, 500 mg can be administered daily over one chemotherapy and/or recovery phase, which can be increased to, e.g., 625 mg daily in a following chemotherapy and/or recovery phase. Reductions in dosage of 5-substituted nucleoside, e.g., BVDU, are also within the scope of the present invention.

The number of doses administered on a daily basis can be reduced by administration of modified release dosage forms. Such forms include without limitation, oral forms (e.g., controlled, extended and/or sustained release dosage forms), and transdermal forms (e.g., extended release patches). Such dosage forms may result in improved patient acceptance and/or compliance. Such dosage forms may permit attaining similar or superior pharmacokinetic parameters (e.g., Tmax, Cmax and/or AUC) compared to instant release dosage forms. Without limitation, modified release dosage forms can be administered, e.g., 1, 2, 3 or 4 times per day.

Any effective pharmacokinetic parameters may be employed with the present invention. Preferred pharmacokinetic parameters provide for pharmaceutically effective levels of cytotoxic agents and of 5-substituted nucleoside. Generally, preferred lower levels of Cmax and AUC are determinable from lowest levels that are pharmaceutically effective. Generally, preferred upper levels of Cmax and AUC are determined by the desire to avoid side-effects, and/or avoid drug wastage. Any Cmax for 5-substituted nucleoside that is pharmaceutically effective is within the scope of the present invention. Without limiting the present invention, preferred Cmax levels for 5-substituted nucleosides are at least about 0.1, 0.5, or 1 μg/ml of blood, and are preferably less than or about 8, 3, or 4 μg/ml. Preferred AUC levels for 5-substituted nucleosides over a 24-hour period are preferably at least about 0.5, 1, or 2 hr·μg/ml, and preferably less than or about 10, 6, or 5 hr·μg/ml.

The cytotoxic agent(s) may remain constant over one or more chemotherapy phases (e.g., may remain the same compounds and/or dosage levels). It is also within the scope of the present invention to vary the compounds and/or dosage levels of cytotoxic agent(s). Such varying may be within a chemotherapy phase or cycle, from one chemotherapy phase or cycle to another, or both. In a preferred embodiment, the dosage of cytotoxic increases within a chemotherapy phase or cycle. When the dosage of cytotoxic agent(s) increases, the dosage of 5-substituted nucleoside, e.g., BVDU, may remain constant or may increase or decrease, and preferably will remain constant.

Without being limited by theory, it is believed that in cultured human pancreatic tumor cells, BVDU inhibits gene products involved in DNA repair such as APEX1, which was also a main effect observed in other tumor cell lines. Treatment with GEM or other cytostatic drugs leads to apurinic sites, which triggers DNA repair, including the induction of APEX1 to restore DNA replication and genetic integrity. Thus, inhibition of DNA repair genes like APEX1 during anticancer treatment increases chemosensitivity, because it was shown that silencing of APEX1 expression by RNA interference enhanced DNA nicking and nearly doubled specific cell lysis.

BVDU downregulates uridine phosphorylase (UPase) expression in BxPC-3 and AsPC-1 human pancreas carcinoma cell lines. It is demonstrated that UPase is highly expressed in a panel of pancreas cancer cell lines and that UPase can potentially be useful in tumor targeting and as a tumor marker. In squamous cell carcinoma, high staining of UPase in primary tumors was frequently associated with the presence of lymph node metastases and lower overall survival. Breast carcinoma patients with high UPase levels had a worse prognosis than those with low levels.

Because of the multiplicity of mechanisms of action it is difficult to understand which mechanism or mechanisms are responsible for the therapeutic effect of the BVDU combination chemotherapy. The numerous immunomodulatory and chemosensitizing properties of BVDU are probably working in concert to optimize chemotherapy and to inhibit chemoresistance.

Without being bound by theory, it is believed that 5-substituted nucleosides can prevent, reduce, or delay induction of chemoresistance and/or poor prognosis, and can do so when administered during and/or after administration of cytotoxics; when cytotoxics are or are not in a patient's system. It is believed that there are several different mechanism by which 5-substituted nucleosides, preferably BVDU (or a prodrug or salt thereof) can accomplish this. It is possible that BVDU (or a prodrug or salt thereof) may act to inhibit overexpression of different survival pathways; of DNA repair genes such as, without limitation, UBE2N and/or APEX1; of oncogenes such as, without limitation, STAT3, DDX1, and/or JUN-D; and/or of uridine phosphorylase (UPase). It is also possible that BVDU (or a prodrug or salt thereof) inhibits overexpression of STAT3 and its target VEGF, potentially leading to activation of anti-tumor immunity by overexpression of lymphotoxins α and β, natural killer cell transcript 4 (NK4), tumor necrosis factor LIGHT/TNFSF-14, ICAM-1; inhibition of tumor proliferation, and/or maintenance of apoptosis. It is also possible that BVDU upregulates DT-diaphorase and/or caspase 3. Without being bound by theory, it is believed that these factors, singly or in combination, can lead to induction of apoptosis and maintenance of chemosensitivity. Maintenance of apoptosis, in turn, can lead to suppression of induces recombination and/or suppression of amplification resistance genes such as, without limitation, MDR and DHFR.

Of concern here are above all the DNA repair genes and/or oncogenes whose expression and/or overexpression can be regulated with a 5-substituted nucleoside. The DNA repair genes and/or oncogenes include, but are not limited to, UBE2N, APEX, DDX1, STAT3, VEGF and/or JUN-D.

Preferably, a 5-substituted nucleoside, prodrug, or salt thereof, is used as overexpression inhibitor.

Preferably, at least one cytostatic in conjunction with at least one overexpression inhibitor of DNA repair gene and/or oncogene or a drug containing the overexpression inhibitor was already used during chemotherapy.

Surprisingly it was able to be shown that, after completion of chemotherapy, if the cells grow further solely with 5-substituted nucleosides (base analogues), the growth thereof is inhibited even more than if the chemotherapy had been continued with cytostatics. Completely unexpectedly, the effect of the 5-substituted nucleosides (base analogues) increases, instead of decreasing, after completion of the chemotherapy phase (treatment).

This effect was established by means of a screening system according to the invention. This screening method is based on the treatment of tumor cells during a chemotherapy cycle over a period of preferably eight to thirty days with increasing doses of a cytostatic and a constant dose of the overexpression inhibitor. After this combination treatment, the cytostatic is discontinued and the treatment is continued solely with the overexpression inhibitor. This recovery phase (also called recovery phase) lasts preferably between 3 and 10 days. Chemotherapy cycles of this type can be implemented successively up to 6 times.

These unexpected results suggest a constellation of treatment forms which would have been surprising for the person skilled in the art:
5-substituted nucleosides, given alone (i.e., not with or after a cytostatic), show no effect.
5-substituted nucleosides, given together with a cytostatic, show an effect.
5-substituted nucleosides, given alone, after they had been given in advance together with a cytostatic (recovery phase), show an increased effect.

Without being bound by theory, it is believed that the observed effect, i.e. the inhibition of chemoresistance and increase in chemosensitivity, can be described as a toxic maintenance of cytostatics-induced apoptosis by influencing the gene expression of specific genes. It is believed that this takes place by:
1) Blockade of “survival pathways” in the recovery phase.
2) Blockade of DNA repair of associated enzymes.
3) Induction of DT-diaphorase activity.
4) Reduced expression of ATP-generated enzymes in the recovery phase.

With respect to 1), base analogues such as BVDU block “survival pathways” principally in the recovery phase of the co-treatment after discontinuing the cytostatics and consequently enforce the course of apoptosis.

By means of HOPI double coloration of AH13r tumor cells of the rat, it was able to be detected that cytostatics such as doxorubicin (DOX), mitoxantrone (MXA) or mitomycin C (MMC) initiate apoptosis. Co-treatment with the base analogue (E)-5-(2-bromovinyl)-2′-deoxyuridine (BVDU) promotes apoptosis by blockade of anti-apoptotic “survival pathways” which include STAT3 and JUN-D.

This effect occurs firstly in the recovery phase of the cells, as can be seen in Example 2.

Constitutively activated STAT3 has an oncogenic effect and contributes to the development of different human cancer diseases. This occurs by inhibition of apoptosis. In this way, STAT3 facilitates the survival of tumor cells and makes cells resistant to chemotherapy. Correspondingly, the inhibition of “STAT3 signaling” induces apoptosis and increases the sensitivity to cytostatics.

JUN-D, a member of the JUN gene family, is an essential component of the “activating protein-1” (AP-1) transcription factor complex with omnipresent expressivity. JUN-D^{(-/-)} primary fibroblasts show premature ageing and increased apoptosis after UV radiation or TNFα treatment. This result leads to the supposition that JUN-D activates the “NFkappaB survival pathway”. Furthermore, p202, which is regulated directly by JUN-D, makes fibroblasts able to resist apoptosis.

Co-treatment by BVDU reduced the expression of both JUN-D isoforms by approximately one quarter. In contrast, STAT3 was regulated in the recovery phase by approximately two thirds (Example 2).

The effect in the recovery phase after co-treatment with mytomycin C is particularly impressive. Here, the base analogue reduces the overexpression of the oncogene JUN-D to the control level (Example 2).

With respect to 2), base analogues such as BVDU block DDX1. DDX1 is co-amplified with MYCN and overexpressed in neuroblastoma (NB) and retinoblastoma cell lines and tumors. NB patients with amplification of both DDX1 and MYCN have a poorer prognosis than patients with only MYCN gene amplification. DDX1 has therefore oncogenic potential.

Co-treatment of MMC with BVDU reduces the overexpression of UBE2N and APEX by approximately one third. Modifications of UBE2N influence the resistance to DNA damage. APEX nuclease is a DNA repair enzyme. Blockade of the APEX expression doubles the cell lysis and increases DNA breakages.

With respect to 3), BVDU induces DT-diaphorase (Example 3). The latter has two properties which are important for the chemotherapy. It activates, on the one hand, cytostatics from the class of quinones and, on the other hand, reduces non-specific toxic effects which are based on the production of reactive oxygen species.

Absence of the DT-D gene leads by reduced p53 and p73 expression to myeloid hyperplasia and correspondingly to reduced apoptosis rates. This is in accord with the observation that a multifactorial “multi-drug resistance” phenotype of tumor cells involves a reduction and no increase in DT-diaphorase expression. Interestingly, the DT-D enzyme activity also stabilizes the lymphocyte populations. This effect could have an advantageous effect on the stabilization of the immune system of patients during chemotherapy.

Many cytostatics, such as e.g. DOX and MXA, disrupt the redox status and the mitochondrial respiration of the cancer cell. This leads to the production of reactive oxygen species (ROS). Not only the cancer cell but also all other cells are affected by the sudden accumulation of ROS, as a result of which undesired side effects occur during chemotherapy.

DT-D inactivates ROS and thus protects cells from non-specific ROS and electrophilic attacks. As an index for this effect of BVDU on the reduction of undesired side effects during chemotherapy, the increase in weight of doxorubicin+BVDU-treated rats may be cited in Example 4. DOX treatment alone leads to weight losses because of the toxic side effects. It is certain that only the side effects (possibly the cardiotoxicity characteristic of DOX) are reduced by BVDU but not the toxic effects on the tumor.

With respect to 4), by altered expression of different enzymes in the recovery phase, the cytostatic effect is maintained also in the absence of a cytostatic. As can be seen in Example 5, the expression of eight genes is increased, that of six genes lowered.

The gene products influence the formation of microfilaments, differentiation, signal transduction and ATP generation.

### EXAMPLES

The invention is further illustrated and described by way of the following non-limiting examples.

### Example 1

BVDU treatment increases the sensitivity of AH13r sarcoma cells to chemotherapy-induced apoptosis. This effect is maintained even after discontinuation of the cytostatic in the so-called recovery phase.

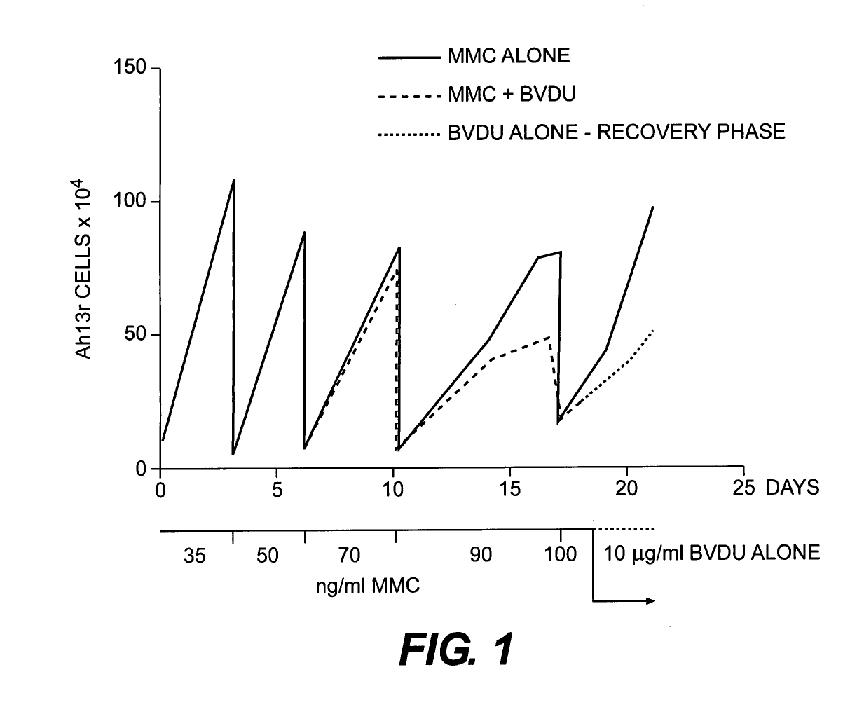

AH13r cells were subjected to increasing doses of the cytostatic mitomycin C (MMC). BVDU, given alone, showed no toxic effect. MMC+BVDU treatment led, after three treatment cycles, to reduction in the cell number in comparison to treatment with MMC alone. This inhibitory effect was maintained even after discontinuation of the cytostatic in the next cycle, in the so-called recovery phase. The cells without MMC and BVDU continued to grow without inhibition. However, those which continued to receive BVDU were greatly inhibited in their growth (see FIG. 1).

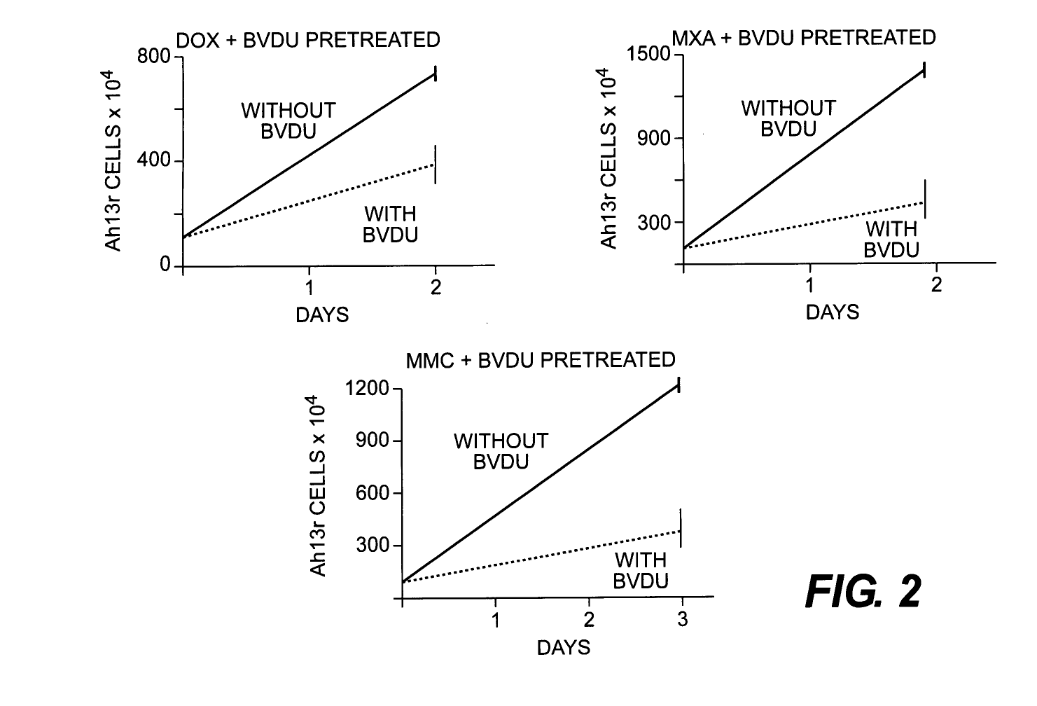

Corresponding results were achieved with methotrexate (MTX), doxorubicin (DOX) and mitoxantrone (MXA) (see FIG. 2).

The indication that the reduction in cell number is based on apoptosis, was detected by means of Hoechst 33258/propidium iodide (Hopi) double coloration.

### Example 2

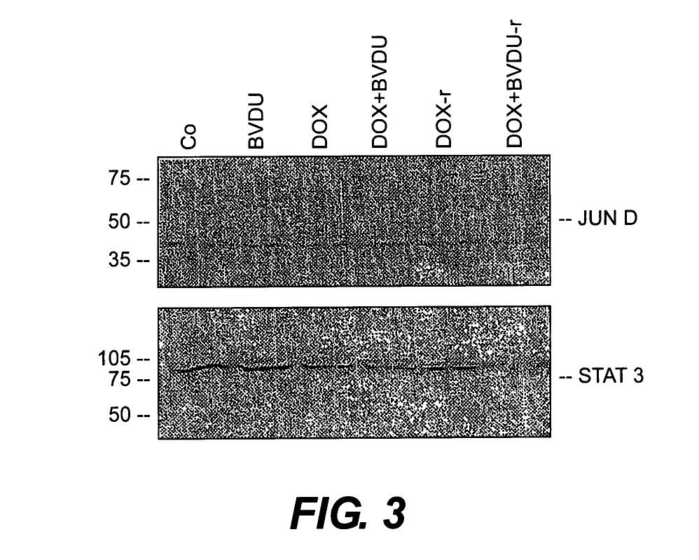

Different “survival pathways” were tested by means of Western Blot analysis. The analyses were implemented according to standard methods, as is described in Sambrook et al., 2001, Molecular Cloning (3^{rd} ed.). Antibody dilutions: P-STAT3 (cell signaling) 1: 500, JUN-D (Santa Cruz, Calif.) 1: 1,000. The upper of the two JUN-D bands shows the “full length isoform” and the lower band the “truncated isoform” which is 48 amino acids shorter. Both isoforms can activate the transcription, but the “full length” variant is more effective than the “truncated” isoform (cf. FIG. 3).

The densitometrically determined content of oncogene proteins JUN-D and STAT3 was reduced by a quarter or two thirds after DOX treatment in the recovery phase (r=recovery phase). In the “recovery” only BVDU is given, no cytostatic.

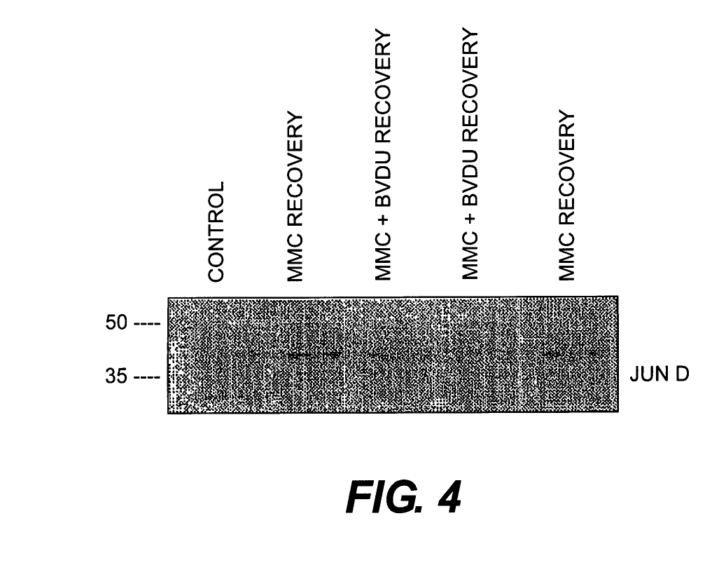

A corresponding result was achieved in the tests with mitomycin C (MMC) (see FIG. 4).

In the test with mitomycin C (MMC), BVDU, given in the “recovery”, effected a complete inhibition of the MMC induced JUN-D overexpression to the control level.

### Example 3

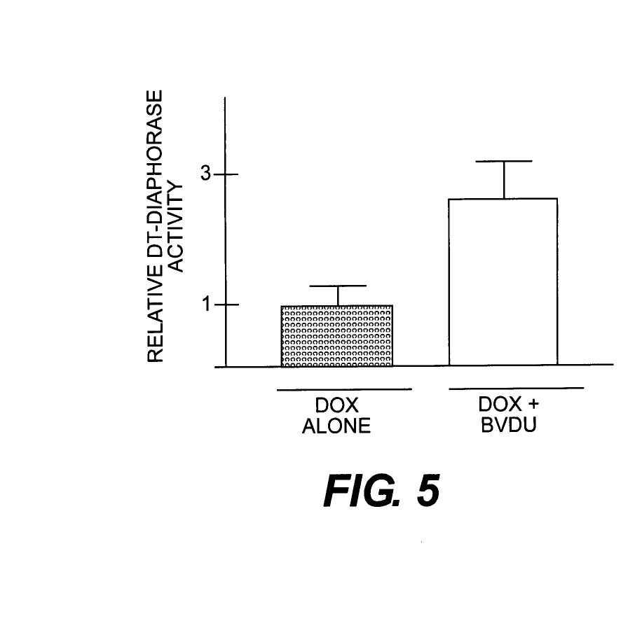

The measurement of the DT-diaphorase (DT-D) was effected as a dicoumarol-inhibitable NAD(P)H: dichlorophenol indophenol reductase, as described in Hodnick et al., Anal. Biochem 252(1), 1997, 165-168 (incorporated by reference herein in its entirety). Extracts were tested of a similar number of cells which had been treated with DOX±BVDU for DT-D activity. Cells treated with BVDU showed an approximately threefold DT-D activity relative to the cells from the control group or from the group of cells treated solely with DOX (see FIG. 5).

Corresponding results were achieved with mitoxantrone (MXA) and methotrexate (MTX). BVDU alone increases the DT-D activity constantly, but in part only weakly.

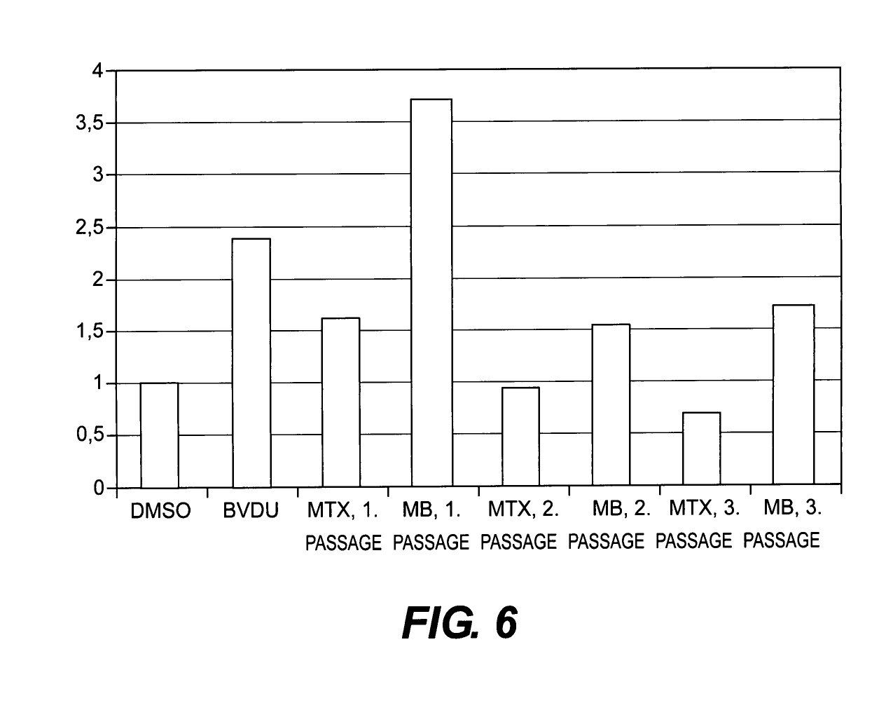

The results with methotrexate (MTX) and human K562 tumor cells are illustrated in FIG. 6, in which “MB” means “MTX and BVDU.” Passage means dilution and conversion of the cells for further growth. The relative DT-D activity is illustrated on the Y axis.

### Example 4

The reduction in toxic side effects of doxorubicin (DOX) was able to be shown in the test with rats (see Table 3). SD rats were treated with dimethybenzanthracene (DMBA). The consequently induced tumors were inhibited in their growth by DOX treatment (1 mg/kg). During the treatment and one day after each treatment, i.e. in the recovery phase, the animals obtained respectively 15 mg/kg BVDU. The recovery phase in vivo was 6× one day.

TABLE 3

| | Relative | | Relative | |
|---|---|---|---|---|
| Average of the | tumor | Relative animal | tumor | Relative animal |
| data from 5-8 | size | weight | size | weight |
| rats | Day 1 | Day 1 | Day 16 | Day 16 |
| Control | 1 | 0 | 6 | +7% |
| DOX alone | 1 | 0 | 1.5 | −7% |
| DOX + BVDU | 1 | 0 | 1 | +7% |

### Example 5

Listing of the proteins influenced by the treatment with base analogues and mitomycin C. The results of the implementation of a two-dimensional gel electrophoresis are compiled in the following Table 4.

TABLE 4

| Protein | DMSO control | BVDU alone |
|---|---|---|
| DEAD/H BOX 1; DDX1 | 0.88 | 0.332 |
| | MMC alone | MMC + BVDU |
| MALATE-DEHYDROGENASE, SOLUBLE; MDH1 | 0.418 | 1.359 |
| MYOSIN, HEAVY CHAIN 1, NORMAL SIMILARITY, ADULT; | 0.182 | 0.588 |
| MYH1 | | |
| UBIQUITIN-CONJUGATING ENZYME E2N; UBE2N | 0.669 | 0.178 |
| APURINIC ENDONUCLEASE; APE; APE1; APEX | 0.363 | 0.14 |
| | MMC “recovery”, | MMC + BVDU |
| | further cultivation | “recovery”, |
| | without MMC and | further treatment by |
| | BVDU | BVDU alone |
| PLATELET-ACTIVATING FACTOR ACETYLHYDROLASE, | 0.219 | 0.619 |
| ISOFORM 1B, ALPHA SUB-UNIT; PAFAH1B1 | | |
| U5 snRNP-SPECIFIC PROTEIN, 116-KD | 0.2 | 0.523 |
| HAEMOGLOBIN-BETA LOCUS; HBB | 0.088 | 0.502 |
| HAEMOGLOBIN-ALPHA LOCUS 1; HBA1 | 0.054 | 0.316 |
| ACTIN, BETA; ACTB | 0.163 | 0.451 |
| Similar to BETA-ACTIN | 0.096 | 0.357 |
| ACTIN similar | 0.112 | 0.398 |
| TROPOMODULIN 2; TMOD2 | 0.095 | 0.28 |
| SUCCINATE-DEHYDROGENASE COMPLEX, SUB-UNIT A, | 0.255 | absent |
| FLAVOPROTEIN; SDHA | | |
| PYRUVATE-DEHYDROGENASE COMPLEX, E1-ALPHA | 1.751 | 0.533 |
| POLYPEPTIDE 1; PDHA1 | | |
| TUBULIN, BETA-5 | 4.705 | 1.553 |
| POLY(rC)-BINDING PROTEIN 2; PCBP2 | 0.912 | 0.234 |
| MALATE-ENZYME 2; ME2 | 0.972 | 0.322 |
| Mini-chromosome preservation inadequate 7; MITOTIN, CELL | 0.374 | 0.119 |
| CLASS CYCLE SIMILAR 1; CDCL1 | | |

### Example 6

Pilot study: 13 Patients (mean age 61, 69% males, 4 stage III, 9 stage IV) with advanced pancreatic adenocarcinoma were treated with i.v. 1.000 mg/m² GEM over 30 min on days 1 and 15 of a 28-day schedule. CIS was administered at 50 mg/m². BVDU treatment took place on the same day and for four days after chemotherapy in the recovery phase (each four 125 mg tablets per day, resulting in a total dose of 6.000 mg per treatment cycle). As control, data were used of patients of a preceding study which were treated with GEM and CIS, but not with BVDU. With the intention to obtain a control group with a similar proportion of stage IV and stage III diseases as the 13 patients of the pilot study, 22 (6 stage III, 15 stage IV) of 96 patients were selected from the Heinemann study by random generator.

BVDU co-treatment enhanced remissions, survival and time to progression (TTP). 77% of the patients lived or have lived longer than one year, and 23% live more than two years. Median survival was 447 days, TTP 280 days, and the response rate 33%.

Dose finding study: 22 Patients with advanced pancreatic adenocarcinoma were eligible for treatment in this single arm study. The mean age was 60 years and 73% of patients were males. One patient without progressive disease was excluded from the study for withdrawal of consent after the first cycle. Of the 21 patients left, 15 were in stage IV and 6 in stage III. BVDU was administered in a combination with GEM. On day 1, on day 8 and on day 15 of each 28-day cycle, BVDU was given together with GEM. On days 2-4, on days 8-11, and on days 15-18 of a 28-day cycle, BVDU was given alone in the recovery phase.

The starting dose of BVDU was 4×125 mg/day (total dose of 6,000 mg per treatment cycle) together with a fixed dose of GEM (1,000 mg/M²). Subsequent total doses of BVDU per cycle were 7,500 mg, 9,000 mg, 10,500 mg, and 12,000 mg again in four patients per cohort. Dosage levels were administered as follows: First dose level: The starting dose of BVDU was 4×125 mg daily given at approximately 08:00 a.m. (1×125 mg BVDU), 12:00 a.m. (1×125 mg BVDU), 04:00 p.m. (1×125 mg BVDU), and 08:00 p.m. (1×125 mg BVDU). These doses were administered on days 1 to 4, days 8 to 11 and days 15 to 18; Second dose level: 1×250 mg+3×125 mg daily; Third dose level. 2×250 mg+2×125 mg daily; Forth dose level: 3×250 mg+1×125 mg daily; and, Fifth dose level: 4×250 mg daily.

Approximately 1 hour after the 08:00 a.m. BVDU-administration on day 1, day 8 and day 15 of a cycle, the GEM infusion started and GEM was infused for 30 minutes.

Patients were treated as long as there was no indication of tumor progression. As control group served a group of 21 patients (Heinemann study) treated with GEM alone, 15 being in stage IV and 6 in stage III of disease, and treated with GEM alone.

Analysis of the concentrations of GEM and BVDU (BVDU) as well as analysis of the concentrations of BVU ((E)-5-(2-bromovinyl)uracil, degradation product of BVDU) in plasma samples: 0.5 ml of plasma spiked with about 0.5 μg of 4-Propyl-2-thiouracil (PTU) as internal standard was extracted with 2.5 ml of methanol-acetonitrile (v/v, 1:9) by vigorous mixing for about 1 min. After centrifugation the supernatant was transferred to a beaker and evaporated to dryness at 50° C. under a gentle stream of nitrogen. The residue was dissolved in 1 ml of the solution used as mobile phase by 10 min treatment in an ultrasonic bath. After centrifugation the clear supernatant was used for HPLC-analysis. The concentrations of GEM and BVDU were calculated using a calibration curve with the respective standard substances. The concentrations of BVU were calculated using the calibration curve of BVDU and the determined response factor. Limits of detection: BVDU 20 ng/ml plasma; GEM 30 ng/ml plasma. Limits of quantification: BVDU 60 ng/ml plasma; GEM 100 ng/ml plasma.

Evaluation: Efficacy was measured by overall survival, objective tumor response rate (ORR, according to WHO criteria in the pilot study and RECIST criteria in the dose finding study) and time to progression (TTP) from the first administration onwards. Serum CA19-9 levels were measured at the start and end of each treatment cycle. Safety was evaluated according to NCI-CTC scale.

Evaluation of Progression: Patients were evaluated every five weeks.

Statistical Methods: Survival curves were generated using the Kaplan-Meier method; survival distributions were compared by the log-rank test (Cox-Mantel), and median survival by the Box and Whisker plot.

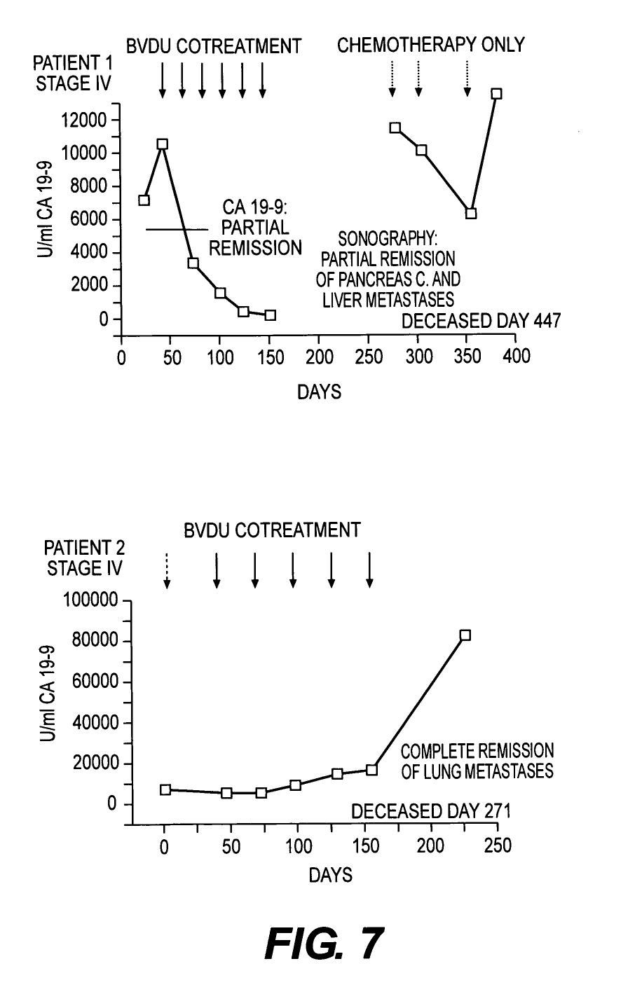

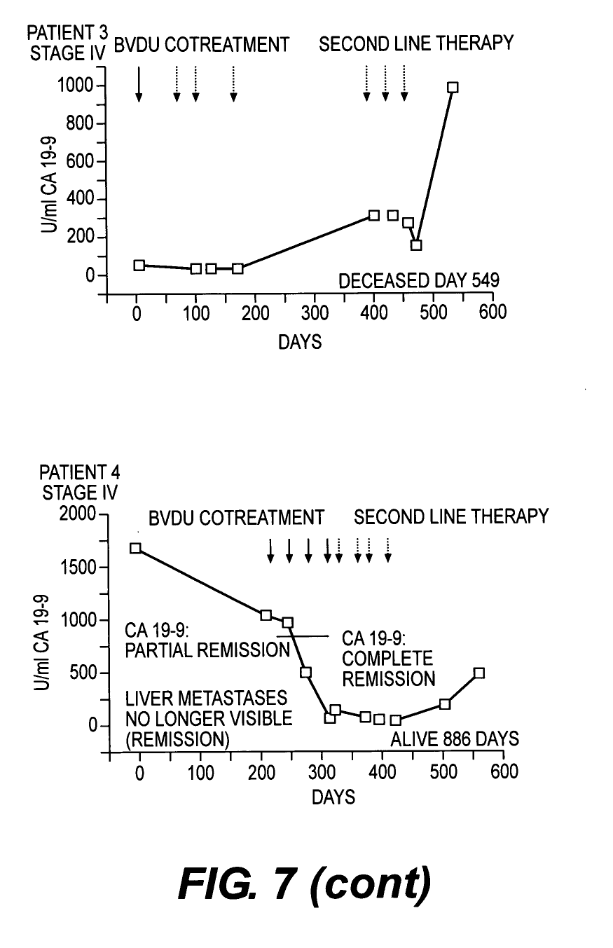

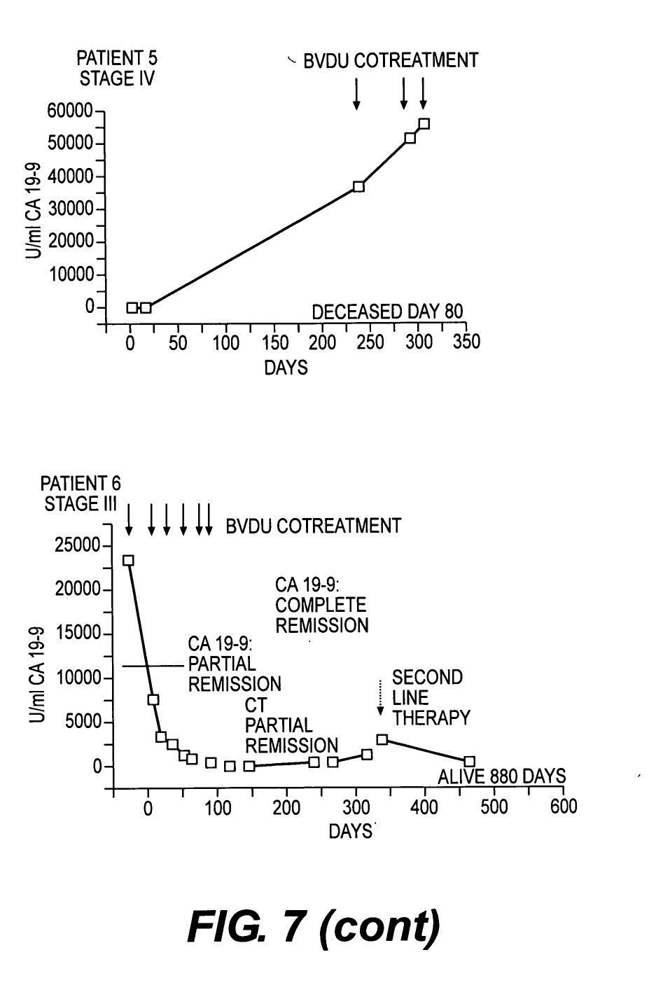

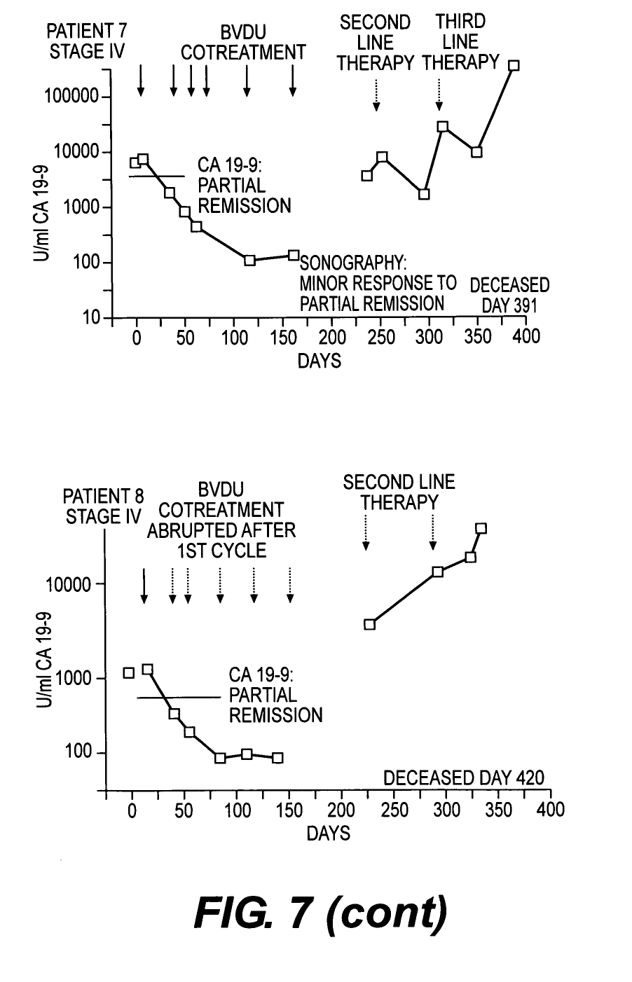

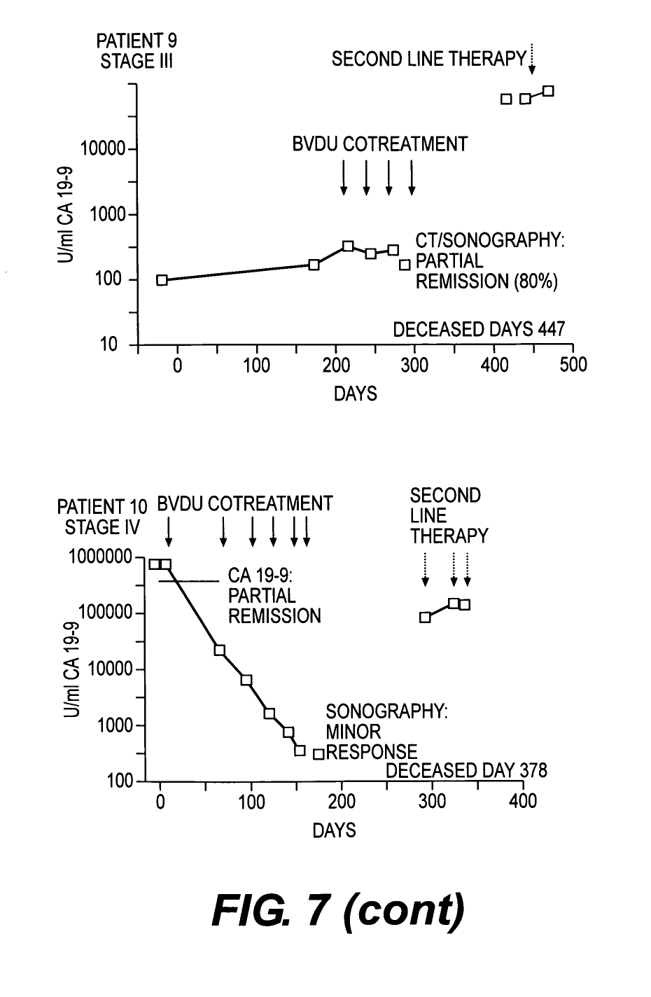

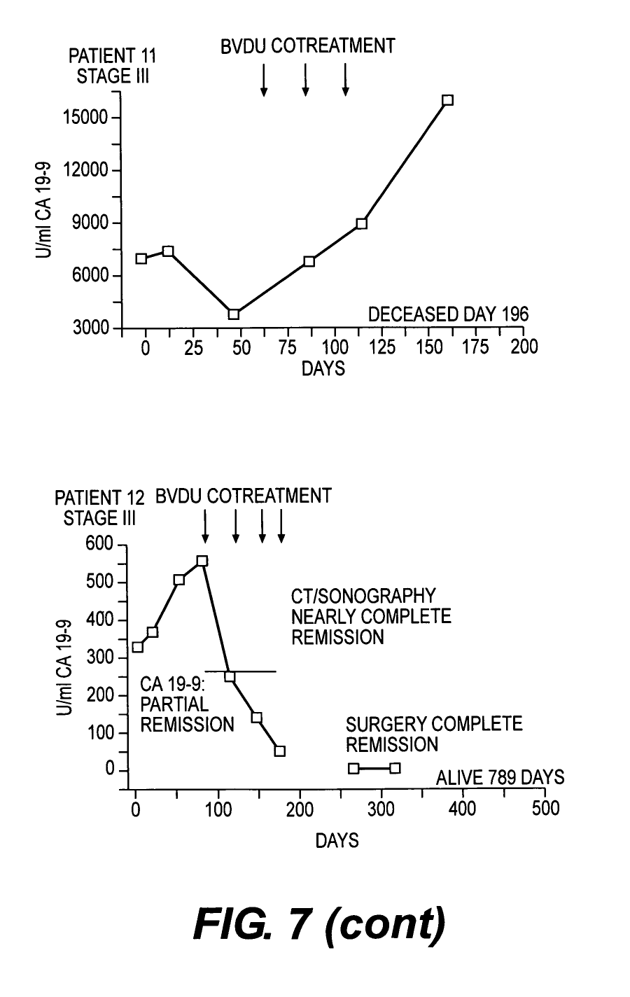

Clinical effects of BVDU in patients treated with GEM+CIS+BVDU: All patients showed at least a stable disease, and 33% of them remissions according to WHO criteria (Table 5). The course of disease was followed by monitoring tumor marker CA19-9. The highest normal value is 35. In 75% of the cases, the tumor marker responded to the co-treatment (total dose of BVDU per cycle was 6.000 mg) and in several cases to the second line therapy without BVDU indicating that the tumors had not acquired resistance (FIG. 7).

TABLE 5
Patients treated with GEM + CIS + BVDU

| | | | | | | | CA19-9 before the |
|---|---|---|---|---|---|---|---|
| | | | | Remissions | | | first and after the |
| Pt | | | Survival | sonography | TTP | Cycle | 6th or earlier |
| no. | age | stage | (days) | and CT | (days) | BVDU | cycle |
| 1 | 52 | TxNxM1G2 | 447 | partial | 279 | 6 | 10621/155 |
| 2 | 59 | TxNxM1G2 | 271 | lung | 238 | 5 | 9372/7678 |
| | | | | metastases | | | |
| 3 | 68 | TxNxM0G4 | 549 | SD | 327 | 1 | 53/25 |
| 4 | 59 | T4NxM1G2 | >886 | Complete: liver | 735 | 4 | 1709/22 |
| | | | | metastases | | | |
| 5 | 53 | T4NxM1G2 | 80 | SD | 70 | 3 | 84/55101 |
| 6 | 63 | TxNxMxG2 | >880 | partial | 281 | 6 | 23362/21 |
| 7 | 63 | T2N0M1Gx | 407 | minor | 247 | 6 | 9436/117 |
| 8 | 64 | TxNxM1G1 | 420 | SD | 331 | 1 | 1101/110 |
| 9 | 66 | T3N1M0G2 | 447 | partial | 184 | 4 | 125/131 |
| 10 | 70 | TxNxM1Gx | 378 | SD | 285 | 6 | 739219/285 |
| 11 | 75 | T4NxM1Gx | 196 | SD | 102 | 3 | 7147/3734 |
| 12 | 41 | T4NxMxG2 | >789 | Partial | >789 | 4 | 332/6 |
| | | | | (complete after | | | |
| | | | | surgery) | | | |
| 13 | 63 | TxNxM1Gx | 463 | SD | n.d. | 2 | n.d. |
| | 61 | 4 × III, 9 × IV | >478 | 33% | >322 | 4 (1-6) | 75% PR or CR |

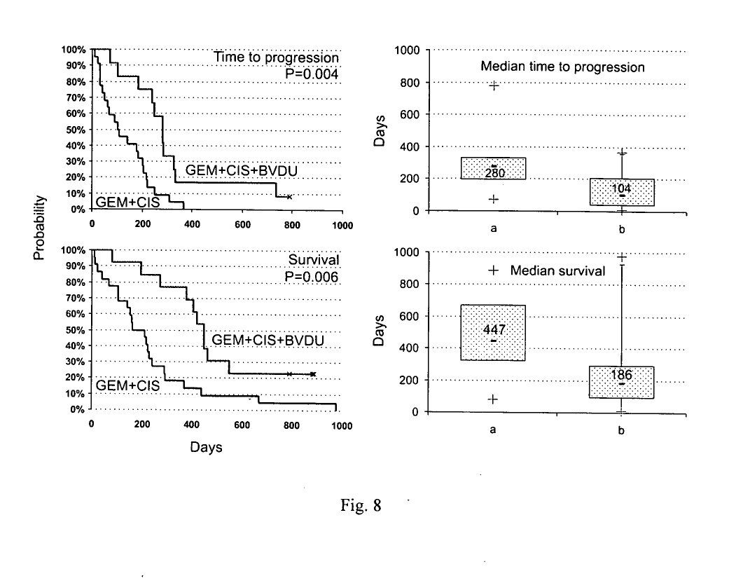

In the BVDU-treatment group, median survival (447 days, p=0.006) and time to progression (280 days, p=0.004) were significantly higher compared to the control group (FIG. 8). Ten of 13 patients (nine in stage IV and four in stage III) lived or live longer than one year after first treatment, and three of them have lived for more than two years by now. The patient characteristics are summarized in Table 5. In the control group, median survival was 186 days and median time to progression 104 days (FIG. 8).

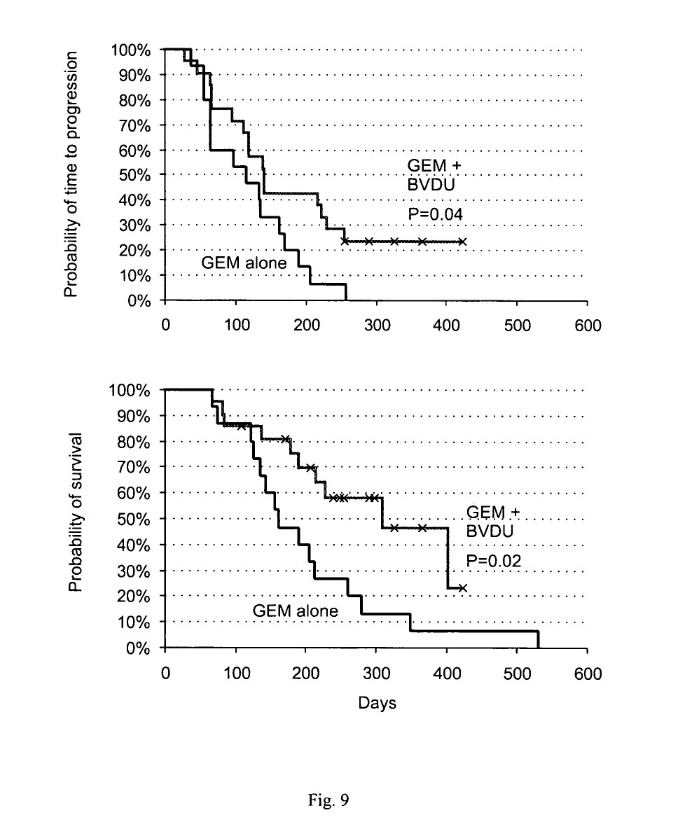

Clinical effects of BVDU in patients treated with GEM+BVDU: Nineteen of 21 patients showed a stable disease, but only one a remission according to RECIST criteria (Table 6). The total doses of BVDU per cycle were 6,000, 7,500 mg, 9,000 mg, 10,500 mg, and 12,000 mg in four patients per group. The results are based on interim data from an ongoing study and patients without progression are still being treated. The data on the survival status show that 15 of 18 (83%) lived or have lived for half a year or longer, and 3 of 9 (33%) for longer than one year. The data of time to progression status show that 9 of 18 (50%) were progression free half a year or longer, and 2 of 9 (22%) one year or longer. The patients being less than half a year, respectively one year in the study were neglected in this calculation. In the control group 8 of 20 patients (40%) lived half a year or longer, and 3 of 20 (15%) one year or longer. Four of 20 (20%) had a TTP half a year or longer. One year TTP was not observed at all. For the analyses according to Kaplan-Meier all BVDU dose groups were combined, which included 21 enrolled patients (FIG. 9). The results are similar but not identical to those of the pilot study with GEM+CIS+BVDU (FIG. 8). To date, adverse events are consistent with those observed with GEM or the underlying disease.

TABLE 6
Patients treated with GEM + BVDU

| | | | | | | | |
|---|---|---|---|---|---|---|---|
| | | | | Remissions | | | |
| Pt | | | Survival | sonography | TTP | Cycle | CA19-9: highest |
| No | Age | Stage | (days) | and CT | days | BVDU | and lowest level |

6,000 mg BVDU per cycle

| | | | | | | | |
|---|---|---|---|---|---|---|---|
| 101 | 60 | T4NxM1G2 | 189 | SD (4,000, | 119 | 5 | 902/142 |
| | | | | 3 × 6,000, 2,000 mg | | | |
| | | | | BVDU) | | | |
| 102 | 68 | T3N1MxG3 | 228 | SD | 139 | 6 | 144/10 |
| 103 | 50 | T4N1M0G1 | 403 | SD | 140 | 6 | 943/256 |
| 104 | 72 | T4N1M1G3 | 178 | SD | 140 | 6 | 12,465/7,229 |

7,500 mg BVDU per cycle

| | | | | | | | |
|---|---|---|---|---|---|---|---|
| 105 | 41 | T3N0M1G2 | >424 | CR | >424 | 8 | 6/3 |
| 106 | 46 | T4N0M0G2 | 214 | SD | 112 | 5 | 98/95 |
| 107 | 73 | T3N0M1G3 | 82 | SD (2 × 7,500, 2,500 mg | 64 | 3 | 19/6 |
| | | | | BVDU) | | | |
| 108 | 62 | T4NxM1G2 | 309 | SD | 217 | 7 | 1,875/494 |
| 201 | 69 | T4N1M1Gx | >366 | SD | >366 | 8+ | 1,640/698 |

9,000 mg BVDU per cycle

| | | | | | | | |
|---|---|---|---|---|---|---|---|
| 109 | 67 | T4N1M0Gx | >326 | SD (9,000, 8,500, | >326 | 6 | 26/18 |
| | | | | 4 × 7.500 mg BVDU) | | | |
| 202 | 69 | T4N1M1G3 | >298 | SD | 255 | 6 | 1,368/490 |
| 110 | 59 | TxNxM1G3 | 137 | SD (3 × 9,000, 6,000 mg | 94 | 4 | 79,200/22,1589 |
| | | | | BVDU) | | | |
| 111 | 56 | T4N1M0Gx | >290 | SD | >290 | 8+ | 3,721/190 |

10,500 mg BVDU per cycle

| 112 | 41 | T3N0M1Gx | 66 | SD | 66 | 2 | 14,000/* |
|---|---|---|---|---|---|---|---|
| 301 | 70 | T4N1M1G1 | >255 | SD (2 × 10,500, | >255 | 6+ | 556/92 |
| | | | | 10,000, 3 × 9,000 mg | | | |
| | | | | BVDU) | | | |
| (302) | (64) | T3N0MxG3 | Excluded: without any progressive | | | [1] | (9,564/7,458) |
| | | | disease cycle 2 not completed | | | | |
| 203 | 68 | TxN0M1Gx | >248 | SD (10,500, 9,500, | 229 | 6 | 7,709/2,973 |
| | | | | 9,000, 7,500) | | | |
| 204 | 62 | T2N1M1G3 | >240 | SD (2 × 10,500, | 223 | 5 | 2,881/1,797 |
| | | | | 9,125, 9,000 mg | | | |
| | | | | BVDU) | | | |

12,000 mg BVDU per cycle

| | | | | | | | |
|---|---|---|---|---|---|---|---|
| 303 | 62 | T3N1M0Gx | >207 | SD (11,500, 9,500 mg | 46 | 2 | 148/357 |
| | | | | BVDU) | | | |
| 205 | 39 | T4N0M1G2 | >171 | SD (12,000, | 119 | 5 | 63,801/8,389 |
| | | | | 11,750, 12,000 mg | | | |
| | | | | BVDU) | | | |
| 206 | 41 | T2N0M1G3 | 83 | PD | 28 | 2 | 13/14 |
| 207 | 67 | T4N0M1G3 | >109 | SD | 65 | 2+ | 218 |

*This value could not be determined because patient survival was too short.

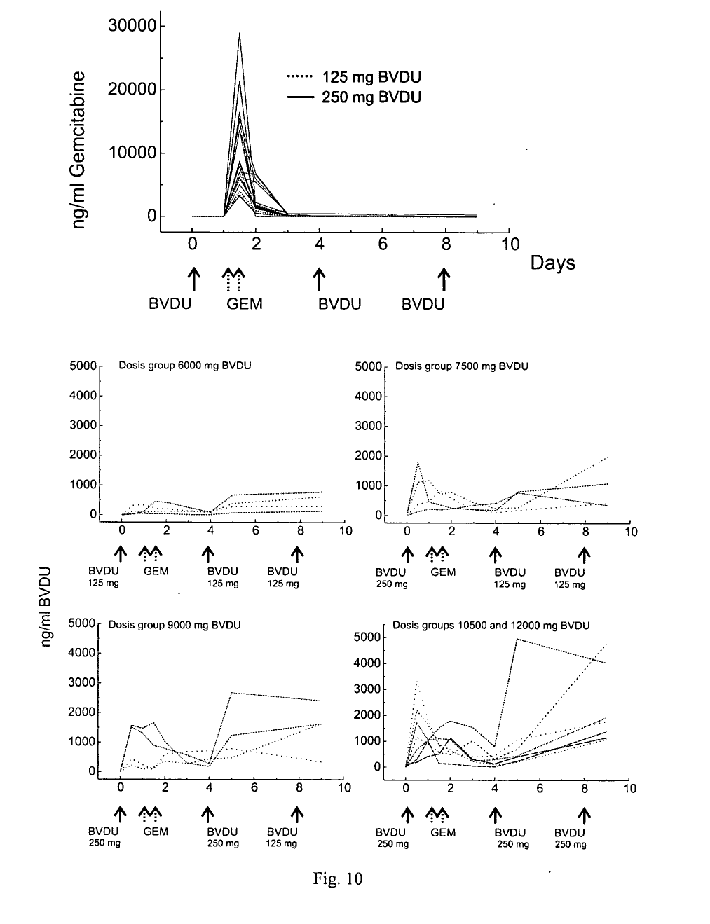

Pharmacokinetic Results BVDU: All measurements were performed within 9 hours. Therefore, only the first three intakes of BVDU-tablets were relevant in this context, and as consequence, in dose groups four and five the intake of tablets was identical. When the dose was doubled from 125 mg to 250 mg, the mean maximum BVDU concentration in plasma increased fivefold from 290 (±152) ng/ml to 1549 (±1058) ng/ml (FIG. 10).

BVU: The mean maximum BVU concentration increased nearly proportional with the dose of BVDU from 1525 (±1012) ng/ml to 4637 (±1353) ng/ml.

GEM: The maximum concentrations were measured immediately after completion of the 30 min infusion. The maximum values varied between 3185 and 29040 ng/ml. BVDU was given half an hour before GEM and the second time 4 hours later. At this time GEM was not detectable any more. Therefore, an influence on GEM concentration by BVDU could only be exerted by the first intake of tablets. For this reason only the first two doses of BVDU (125 and 250 mg) can be compared. The GEM maximum values appear to rise with increase of the BVDU dose but the large degree of variability between individual patients should be considered (FIG. 10).

### Example 7

Ten SD-rats per treatment group were given a single s.c. injection of ascites Yoshida AH13r hepatoma cells. Five to 7 days after tumor application, the growth of the resulting tumors (at the injection site) was suppressed by i.p. treatment of the animals with cisplatin, doxorubicin or cyclophosphamide, or by additional oral treatment with 15 mg/kg BVDU (15 times within 3 weeks).

As seen in FIG. 12, BVDU, given alone, exerts no influence on the growths of rat tumors. On the contrary, the tumors seem to grow rather faster than slower than those without treatment.

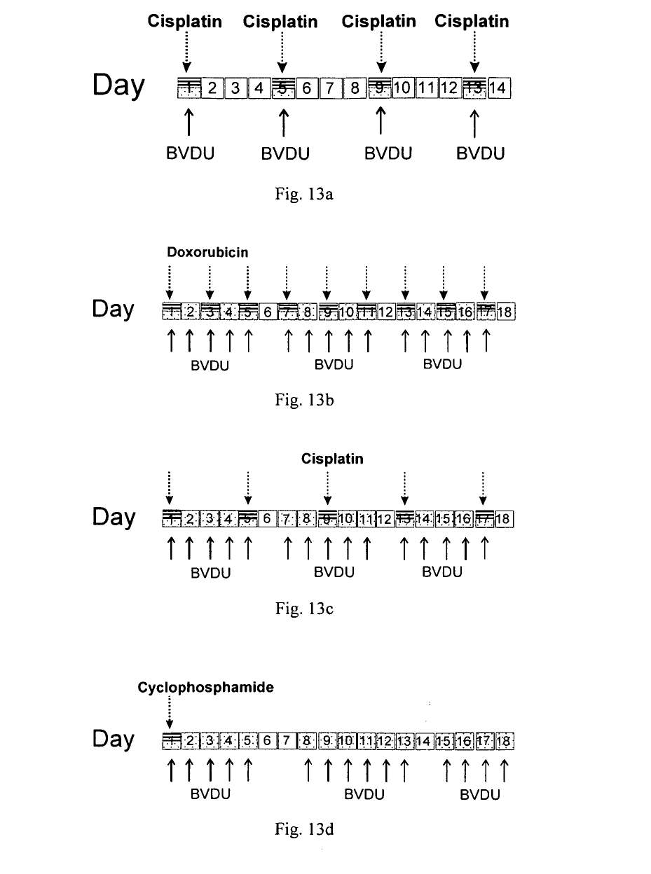

The result of chemotherapy without “recovery” effect is shown in the treatment schedule of FIG. 13a. In this instance, BVDU is given simultaneously with cisplatin (CIS):

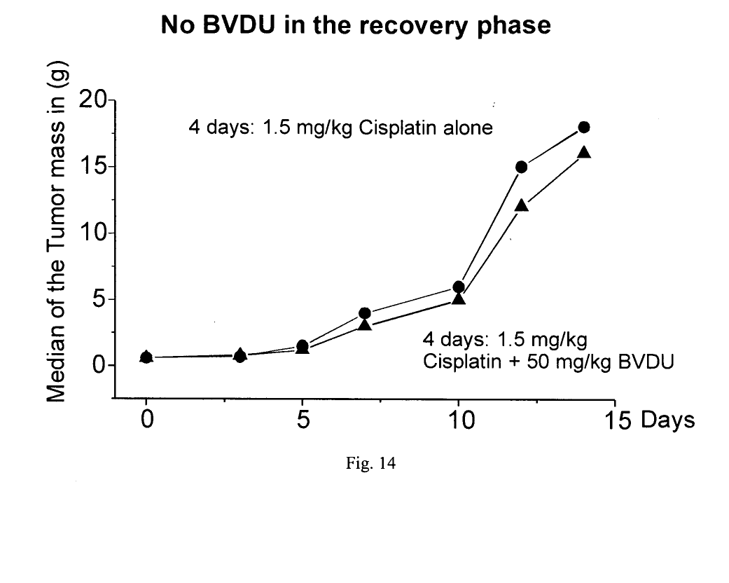

FIG. 14 shows the effect of BVDU if given only at the same day as the cytotoxic drug, and indicates that if BVDU is given only on the same day as the cytotoxic drug, its effect is only weak. In these experiments the dose was very high, i.e. 50 mg/kg. This is much more than the standard dose of 15 mg/kg.

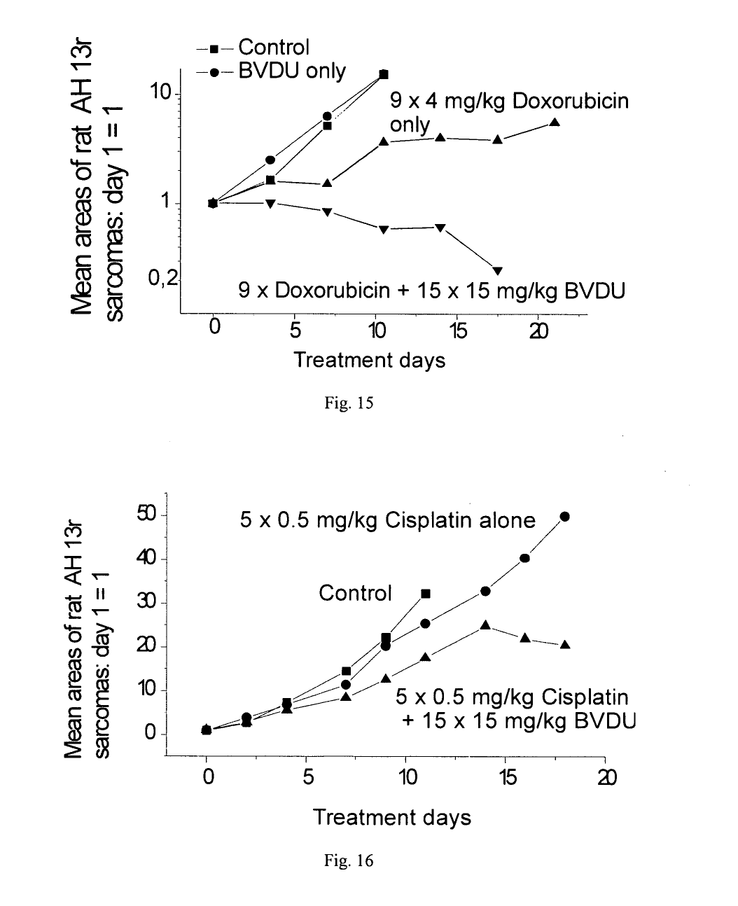

As shown in FIG. 13b, doxorubicin may be given to provide a recovery phase in vivo of 6× one day. FIG. 15 shows the effect of this treatment schedule of BVDU in the “recovery” phase. BVDU, if given rotatory alone or in combination with DOX in the “recovery” phase, has a strong effect. The effect of BVDU is very strong, even if the recovery phase is just only one day.

The recovery phase can be enhanced to 4×3 or 2 days, obtaining a treatment schedule such as shown in FIG. 13c. The effect of this treatment schedule is shown in FIG. 16, showing that the effect of BVDU is very strong, especially when compared to the very weak effect if the treatment is restricted to an exclusively simultaneous administration of CIS+BVDU (e.g., FIG. 14).

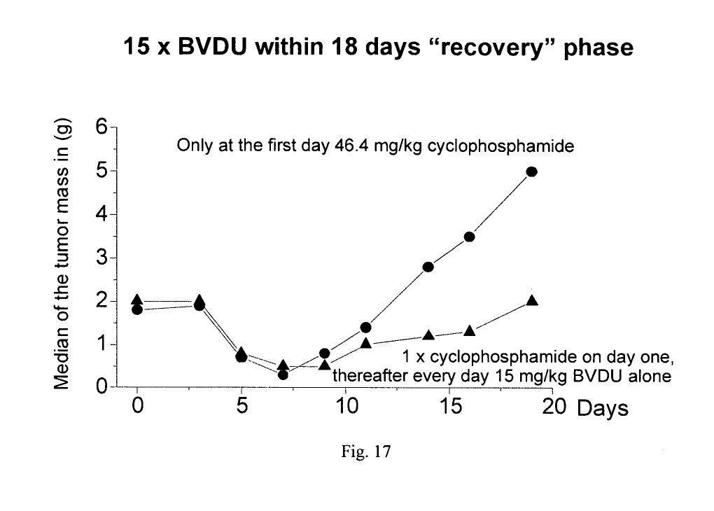

An elongation of the treatment in the recovery phase is preferred with cyclophosphamide (FIG. 13d), as cyclophosphamide has sufficient long term effects. The effect of 14 days treatment with BVDU in the “recovery” phase is shown in FIG. 17, indicating that BVDU, given 15 times alone in the recovery phase and only one time at the beginning of treatment together with cyclophosphamide, has a strong effect.

Cyclophosphamide gives the best example for the effect of BVDU in the recovery phase. In summary, BVDU allows different treatment schedules for different cytotoxic drugs. The recovery treatment phase can vary from 6× one day to 1×14 days.

The practical application of these results of animal experiments can be seen in the clinical studies of patients with pancreatic cancer.

Although the present invention has been described in considerable detail with regard to certain versions thereof, other versions are possible, and alterations, permutations, and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

We claim:

1. A method for treating a cancer treatable with gemcitabine comprising administering to a patient in need thereof a therapeutically effective amount of a cytotoxic composition comprising gemcitabine in a chemotherapy phase, and a therapeutically effective amount of a 5-substituted nucleoside, prodrug or salt thereof, or combination of two or more thereof, in at least one of the chemotherapy phase and a recovery phase.

2. The method of claim 1 wherein the cancer treatable with gemcitabine includes pancreatic cancer.

3. The method of claim 1 comprising administering the 5-substituted nucleoside, prodrug or salt thereof, or combination of two or more thereof, during the chemotherapy phase.

4. The method of claim 3 wherein the chemotherapy phase is about 1 to 5 days.

5. The method of claim 1 comprising administering the 5-substituted nucleoside, prodrug or salt thereof, or combination of two or more thereof, during the recovery phase.

6. The method of claim 5 wherein the recovery phase is about 1 to 10 days.

7. The method of claim 5 further comprising administering a 5-substituted nucleoside, prodrug or salt thereof, or combination of two or more thereof, during the chemotherapy phase.

8. The method of claim 7 wherein the chemotherapy phase is about 1 to 30 days.

9. The method of claim 8 wherein the recovery phase is about 1 to 30 days.

10. The method of claim 9 further comprising a rest phase during which no cytotoxic agent or 5-substituted nucleoside is administered.

11. The method of claim 10 wherein the rest phase is about 8 to 60 days.

12. The method of claim 1 wherein the cytotoxic composition comprises a second cytotoxic agent other than gemcitabine.

13. The method of claim 12 wherein the second cytotoxic agent comprises a platinum compound.

14. The method of claim 13 wherein the second cytotoxic agent comprises cisplatin.

15. The method of claim 1 wherein the 5-substituted nucleoside, prodrug, or salt thereof, or combination of two or more thereof, comprises BVDU, or a prodrug, or salt thereof.

16. The method of claim 15 wherein the patient is administered the BVDU, prodrug, or salt thereof during the chemotherapy phase, and attains a BVDU blood level of about 0.02 to 50 μg/ml during the chemotherapy phase.

17. The method of claim 15 wherein the patient is administered the BVDU, prodrug, or salt thereof, during the chemotherapy phase, and attains a BVDU AUC of at least about 0.5 μg·hr/ml over a 24-hour period during the chemotherapy phase.

18. The method of claim 15 wherein the patient is administered the BVDU, prodrug, or salt thereof during the recovery phase, and attains a BVDU blood level of about 0.02 to 50 μg/ml during the recovery phase.

19. The method of claim 18 wherein the patient is administered the BVDU, prodrug, or salt thereof during the chemotherapy phase, and attains a BVDU blood level of about 0.02 to 50 μg/ml during the chemotherapy phase.

20. The method of claim 15 wherein the patient is administered the BVDU, or prodrug, or salt thereof during the chemotherapy phase, and attains a BVDU AUC of at least about 0.5 μg·hr/ml over a 24-hour period during the recovery phase.

21. The method of claim 20 wherein the patient is administered the BVDU, prodrug, or salt thereof during the chemotherapy phase, and attains a BVDU AUC of at least about 0.5 μg·hr/ml over a 24-hour period during the chemotherapy phase.

22. The method of claim 15 wherein the BVDU, prodrug or salt thereof comprises the prodrug represented by the formula I: 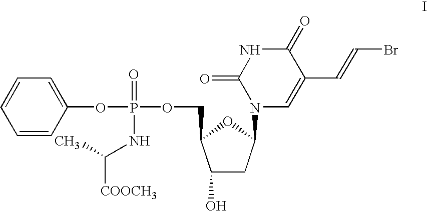
or a salt thereof.

23. A method for reducing chemoresistance comprising administering to a patient in need thereof a therapeutically effective amount of gemcitabine in a chemotherapy phase, and a therapeutically effective amount of a 5-substituted nucleoside, a prodrug or salt thereof, or combination of two or more thereof, in at least one of the chemotherapy phase or a recovery phase.

24. A method for enhancing chemosensitivity comprising administering to a patient in need thereof a therapeutically effective amount of gemcitabine in a chemotherapy phase, and a therapeutically effective amount of a 5-substituted nucleoside, a prodrug or salt thereof, or combination of two or more thereof, in at least one of the chemotherapy phase or a recovery phase.

25. A method for enhancing the cytotoxic effect of gemcitabine in treatment of pancreatic cancer, comprising administering a therapeutically effective amount of gemcitabine to a cancer patient in a chemotherapy phase, and a therapeutically effective amount of BVDU, or a prodrug, or salt thereof, in a recovery phase following the chemotherapy phase.
